SEQUENCE LISTING (278 sequences)

SEQ ID NO: 1 - DNA, 22 bp, artificial sequence
  laboratory synthesized sequence
cagttctccctgaagctgagct

SEQ ID NO: 2 - DNA, 21 bp, artificial sequence
  laboratory synthesized sequence
cagcctacatggagctgagca

SEQ ID NO: 3 - DNA, 24 bp, artificial sequence
  laboratory synthesized sequence
acacgctgtatctgcaaatgaaca

SEQ ID NO: 4 - DNA, 26 bp, artificial sequence
  laboratory synthesized sequence
gaactcactgtatctgcaaatgaaca

SEQ ID NO: 5 - DNA, 26 bp, artificial sequence
  laboratory synthesized sequence
gaacacgctgtatcttcaaatgaaca

SEQ ID NO: 6 - DNA, 25 bp, artificial sequence
  laboratory synthesized sequence
ctccctgtatctgcaaatgaacagt

SEQ ID NO: 7 - DNA, 24 bp, artificial sequence
  laboratory synthesized sequence
caggtggtccttacaatgaccaac

SEQ ID NO: 8 - DNA, 19 bp, artificial sequence
  laboratory synthesized sequence
gcctacctgcagtggagca

SEQ ID NO: 9 - DNA, 20 bp, artificial sequence
  laboratory synthesized sequence
gcagacacggccgtgtatta

SEQ ID NO: 10 - DNA, 22 bp, artificial sequence
  laboratory synthesized sequence
gcacggcatatctgcagatctg

SEQ ID NO: 11 - DNA, 23 bp, artificial sequence
  laboratory synthesized sequence
gagctgaggacacggctgtgtat

SEQ ID NO: 12 - DNA, 23 bp, artificial sequence
  laboratory synthesized sequence
gacacagcctacatggagctgag

SEQ ID NO: 13 - DNA, 24 bp, artificial sequence
  laboratory synthesized sequence
gaacacgctgcatcttcaaatgaa

SEQ ID NO: 14 - DNA, 21 bp, artificial sequence
  laboratory synthesized sequence
cggcgtatctgcaaatgaaca

SEQ ID NO: 15 - DNA, 21 bp, artificial sequence
  laboratory synthesized sequence
cgctgtatcttcaaatgggca

SEQ ID NO: 16 - DNA, 23 bp, artificial sequence
  laboratory synthesized sequence
catcgcctatctgcaaatgaaca

SEQ ID NO: 17 - DNA, 22 bp, artificial sequence
  laboratory synthesized sequence
cagttctccctgcagctgaact

SEQ ID NO: 18 - DNA, 21 bp, artificial sequence
  laboratory synthesized sequence
cactgcctacctgcagtggag

SEQ ID NO: 19 - DNA, 21 bp, artificial sequence
  laboratory synthesized sequence
cacggcatatctgcagatctg

SEQ ID NO: 20 - DNA, 23 bp, artificial sequence
  laboratory synthesized sequence
cacagtctacatggagctgagca

SEQ ID NO: 21 - DNA, 29 bp, artificial sequence
  laboratory synthesized sequence
caagaactccttgtatcttcaaatgaaca

SEQ ID NO: 22 - DNA, 23 bp, artificial sequence
  laboratory synthesized sequence
aggtggtccttaccatgaccaac

SEQ ID NO: 23 - DNA, 25 bp, artificial sequence
  laboratory synthesized sequence
agaacacgctgtatctgcaaatgaa

SEQ ID NO: 24 - DNA, 22 bp, artificial sequence
  laboratory synthesized sequence
acagcctacatggagctgagga

SEQ ID NO: 25 - DNA, 28 bp, artificial sequence
  laboratory synthesized sequence
aagaactcactgtatctgcaaatgaaca

SEQ ID NO: 26 - DNA, 25 bp, artificial sequence
  laboratory synthesized sequence
aactccctgtatctgcaaatgaaca

SEQ ID NO: 27 - DNA, 25 bp, artificial sequence
  laboratory synthesized sequence
aacacgctgtatctgcaaatgaaca

SEQ ID NO: 28 - DNA, 19 bp, artificial sequence
  laboratory synthesized sequence
acctgaggagacggtgacc

SEQ ID NO: 29 - DNA, 21 bp, artificial sequence
  laboratory synthesized sequence
gaccttggtcccagttccgaa

SEQ ID NO: 30 - DNA, 18 bp, artificial sequence
  laboratory synthesized sequence
cttggtccctccgccgaa

SEQ ID NO: 31 - DNA, 18 bp, artificial sequence
  laboratory synthesized sequence
cttggtccctccgccgaa

SEQ ID NO: 32 - DNA, 19 bp, artificial sequence
  laboratory synthesized sequence
ccttggtgccactgccgaa

SEQ ID NO: 33 - DNA, 19 bp, artificial sequence
  laboratory synthesized sequence
gctgggtgcctcctccgaa

SEQ ID NO: 34 - DNA, 22 bp, artificial sequence
  laboratory synthesized sequence
gggctccagtctgaggatgagg

SEQ ID NO: 35 - DNA, 22 bp, artificial sequence
  laboratory synthesized sequence
gggctccaggctgaggatgagg

SEQ ID NO: 36 - DNA, 22 bp, artificial sequence
  laboratory synthesized sequence
gggctccagtctgaggatgagg

SEQ ID NO: 37 - DNA, 19 bp, artificial sequence
  laboratory synthesized sequence
ctccggtccgaggatgagg

SEQ ID NO: 38 - DNA, 21 bp, artificial sequence
  laboratory synthesized sequence
gactccagactggggacgagg

SEQ ID NO: 39 - DNA, 20 bp, artificial sequence
  laboratory synthesized sequence
gggctccaggctgaggatga

SEQ ID NO: 40 - DNA, 31 bp, artificial sequence
  laboratory synthesized sequence
tgaccatctctgggctccaggctgaggatga

SEQ ID NO: 41 - DNA, 19 bp, artificial sequence
  laboratory synthesized sequence
ggctccaggctgaggacga

SEQ ID NO: 42 - DNA, 20 bp, artificial sequence
  laboratory synthesized sequence
agcgggacccaggctatgga

SEQ ID NO: 43 - DNA, 17 bp, artificial sequence
  laboratory synthesized sequence
cagagcccaagccgggg

SEQ ID NO: 44 - DNA, 20 bp, artificial sequence
  laboratory synthesized sequence
ggcccaggtggaggatgaag

SEQ ID NO: 45 - DNA, 20 bp, artificial sequence
  laboratory synthesized sequence
gatcgaggctggggatgagg

SEQ ID NO: 46 - DNA, 22 bp, artificial sequence
  laboratory synthesized sequence
tgagtccaggcagaagacgagg

SEQ ID NO: 47 - DNA, 20 bp, artificial sequence
  laboratory synthesized sequence
gctcaggcggaagatgaggc

SEQ ID NO: 48 - DNA, 17 bp, artificial sequence
  laboratory synthesized sequence
gaagccggggatgaggc

SEQ ID NO: 49 - DNA, 20 bp, artificial sequence
  laboratory synthesized sequence
cagcagggtcctgaccgaag

SEQ ID NO: 50 - DNA, 21 bp, artificial sequence
  laboratory synthesized sequence
cagaagatgaggctgactatt

SEQ ID NO: 51 - DNA, 17 bp, artificial sequence
  laboratory synthesized sequence
cggggcccaggttgagg

SEQ ID NO: 52 - DNA, 26 bp, artificial sequence
  laboratory synthesized sequence
ccagtctgacgatgaggctgagtatc

SEQ ID NO: 53 - DNA, 24 bp, artificial sequence
  laboratory synthesized sequence
ccatctccaacctccagtttgagg

SEQ ID NO: 54 - DNA, 23 bp, artificial sequence
  laboratory synthesized sequence
catctccagcctccagtctgagg

SEQ ID NO: 55 - DNA, 19 bp, artificial sequence
  laboratory synthesized sequence
ccgggctccagtctgagga

SEQ ID NO: 56 - DNA, 24 bp, artificial sequence
  laboratory synthesized sequence
catctctgggctccagtctgaaga

SEQ ID NO: 57 - DNA, 23 bp, artificial sequence
  laboratory synthesized sequence
atctctgggctccagtctgagga

SEQ ID NO: 58 - DNA, 20 bp, artificial sequence
  laboratory synthesized sequence
gctccagcctgaggatgagg

SEQ ID NO: 59 - DNA, 25 bp, artificial sequence
  laboratory synthesized sequence
ccatctctggactgaagactgagga

SEQ ID NO: 60 - DNA, 21 bp, artificial sequence
  laboratory synthesized sequence
gtcaggtgtgcagcctgagga

SEQ ID NO: 61 - DNA, 17 bp, artificial sequence
  laboratory synthesized sequence
ggtgcgcagcctgagga

SEQ ID NO: 62 - DNA, 18 bp, artificial sequence
  laboratory synthesized sequence
gggcccaggcagatgatg

SEQ ID NO: 63 - DNA, 26 bp, artificial sequence
  laboratory synthesized sequence
ccaggaagaagatgagagtgactacc

SEQ ID NO: 64 - DNA, 21 bp, artificial sequence
  laboratory synthesized sequence
gactccagcctgaggacgagg

SEQ ID NO: 65 - DNA, 55 bp, artificial sequence
  laboratory synthesized sequence
cccgagctcatctggcataattctcctagttttccagcctacatggagctgagca

SEQ ID NO: 66 - DNA, 55 bp, artificial sequence; identical to SEQ ID NO: 65
  laboratory synthesized sequence

SEQ ID NO: 67 - DNA, 55 bp, artificial sequence; identical to SEQ ID NO: 65
  laboratory synthesized sequence

SEQ ID NO: 68 - DNA, 57 bp, artificial sequence
  laboratory synthesized sequence
cccgagctcatctggcataattctcctagttttcgacacagcctacatggagctgag

SEQ ID NO: 69 - DNA, 56 bp, artificial sequence
  laboratory synthesized sequence
cccgagctcatctggcataattctcctagttttcacagcctacatggagctgagga

SEQ ID NO: 70 - DNA, 55 bp, artificial sequence; identical to SEQ ID NO: 65
  laboratory synthesized sequence

SEQ ID NO: 71 - DNA, 55 bp, artificial sequence; identical to SEQ ID NO: 65
  laboratory synthesized sequence

SEQ ID NO: 72 - DNA, 57 bp, artificial sequence
  laboratory synthesized sequence
cccgagctcatctggcataattctcctagttttccacagtctacatggagctgagca

SEQ ID NO: 73 - DNA, 55 bp, artificial sequence; identical to SEQ ID NO: 65
  laboratory synthesized sequence

SEQ ID NO: 74 - DNA, 55 bp, artificial sequence; identical to SEQ ID NO: 65
  laboratory synthesized sequence

SEQ ID NO: 75 - DNA, 58 bp, artificial sequence
  laboratory synthesized sequence
cccgagctcatctggcataattctcctagttttccaggtggtccttacaatgaccaac

SEQ ID NO: 76 - DNA, 57 bp, artificial sequence
  laboratory synthesized sequence
cccgagctcatctggcataattctcctagttttcaggtggtccttaccatgaccaac

SEQ ID NO: 77 - DNA, 58 bp, artificial sequence; identical to SEQ ID NO: 75
  laboratory synthesized sequence

SEQ ID NO: 78 - DNA, 58 bp, artificial sequence
  laboratory synthesized sequence
cccgagctcatctggcataattctcctagttttcgaacacgctgcatcttcaaatgaa

SEQ ID NO: 79 - DNA, 57 bp, artificial sequence
  laboratory synthesized sequence
cccgagctcatctggcataattctcctagttttcgagctgaggacacggctgtgtat

SEQ ID NO: 80 - DNA, 59 bp, artificial sequence
  laboratory synthesized sequence
cccgagctcatctggcataattctcctagttttcctccctgtatctgcaaatgaacagt

SEQ ID NO: 81 - DNA, 62 bp, artificial sequence
  laboratory synthesized sequence
cccgagctcatctggcataattctcctagttttcaagaactcactgtatctgcaaatgaaca

SEQ ID NO: 82 - DNA, 63 bp, artificial sequence
  laboratory synthesized sequence
cccgagctcatctggcataattctcctagttttccaagaactccttgtatcttcaaatgaaca

SEQ ID NO: 83 - DNA, 59 bp, artificial sequence
  laboratory synthesized sequence
cccgagctcatctggcataattctcctagttttcaacacgctgtatctgcaaatgaaca

SEQ ID NO: 84 - DNA, 59 bp, artificial sequence
  laboratory synthesized sequence
cccgagctcatctggcataattctcctagttttcaactccctgtatctgcaaatgaaca

SEQ ID NO: 85 - DNA, 60 bp, artificial sequence
  laboratory synthesized sequence
cccgagctcatctggcataattctcctagttttcgaactcactgtatctgcaaatgaaca

SEQ ID NO: 86 - DNA, 58 bp, artificial sequence
  laboratory synthesized sequence
cccgagctcatctggcataattctcctagttttcacacgctgtatctgcaaatgaaca

SEQ ID NO: 87 - DNA, 58 bp, artificial sequence; identical to SEQ ID NO: 86
  laboratory synthesized sequence

SEQ ID NO: 88 - DNA, 59 bp, artificial sequence
  laboratory synthesized sequence
cccgagctcatctggcataattctcctagttttcagaacacgctgtatctgcaaatgaa

SEQ ID NO: 89 - DNA, 58 bp, artificial sequence
  laboratory synthesized sequence
acccgagctcatctggcataattctcctagttttccacgctgtatctgcaaatgaaca

SEQ ID NO: 90 - DNA, 59 bp, artificial sequence; identical to SEQ ID NO: 80
  laboratory synthesized sequence

SEQ ID NO: 91 - DNA, 60 bp, artificial sequence
  laboratory synthesized sequence
gcccgagctcatctggcataattctcctagttttcaactcactgtatctgcaaatgaaca

SEQ ID NO: 92 - DNA, 57 bp, artificial sequence
  laboratory synthesized sequence
cccgagctcatctggcataattctcctagttttccatcgcctatctgcaaatgaaca

SEQ ID NO: 93 - DNA, 60 bp, artificial sequence
  laboratory synthesized sequence
cccgagctcatctggcataattctcctagttttcgaacacgctgtatcttcaaatgaaca

SEQ ID NO: 94 - DNA, 55 bp, artificial sequence
  laboratory synthesized sequence
cccgagctcatctggcataattctcctagttttccgctgtatcttcaaatgggca

SEQ ID NO: 95 - DNA, 60 bp, artificial sequence; identical to SEQ ID NO: 93
  laboratory synthesized sequence

SEQ ID NO: 96 - DNA, 60 bp, artificial sequence; identical to SEQ ID NO: 85
  laboratory synthesized sequence

SEQ ID NO: 97 - DNA, 55 bp, artificial sequence
  laboratory synthesized sequence
cccgagctcatctggcataattctcctagttttccggcgtatctgcaaatgaaca

SEQ ID NO: 98 - DNA, 58 bp, artificial sequence; identical to SEQ ID NO: 86
  laboratory synthesized sequence

SEQ ID NO: 99 - DNA, 55 bp, artificial sequence
  laboratory synthesized sequence
cccgagctcatctggcataattctcctagttttccacggcatatctgcagatctg

SEQ ID NO: 100 - DNA, 56 bp, artificial sequence
  laboratory synthesized sequence
cccgagctcatctggcataattctcctagttttccagttctccctgaagctgagct

SEQ ID NO: 101 - DNA, 56 bp, artificial sequence; identical to SEQ ID NO: 100
  laboratory synthesized sequence

SEQ ID NO: 102 - DNA, 56 bp, artificial sequence; identical to SEQ ID NO: 100
  laboratory synthesized sequence

SEQ ID NO: 103 - DNA, 54 bp, artificial sequence
  laboratory synthesized sequence
cccgagctcatctggcataattctcctagttttcgcagacacggccgtgtatta

SEQ ID NO: 104 - DNA, 56 bp, artificial sequence; identical to SEQ ID NO: 100
  laboratory synthesized sequence

SEQ ID NO: 105 - DNA, 56 bp, artificial sequence; identical to SEQ ID NO: 100
  laboratory synthesized sequence

SEQ ID NO: 106 - DNA, 56 bp, artificial sequence; identical to SEQ ID NO: 100
  laboratory synthesized sequence

SEQ ID NO: 107 - DNA, 56 bp, artificial sequence; identical to SEQ ID NO: 100
  laboratory synthesized sequence

SEQ ID NO: 108 - DNA, 56 bp, artificial sequence; identical to SEQ ID NO: 100
  laboratory synthesized sequence

SEQ ID NO: 109 - DNA, 55 bp, artificial sequence
  laboratory synthesized sequence
cccgagctcatctggcataattctcctagttttccactgcctacctgcagtggag

SEQ ID NO: 110 - DNA, 53 bp, artificial sequence
  laboratory synthesized sequence
cccgagctcatctggcataattctcctagttttcgcctacctgcagtggagca

SEQ ID NO: 111 - DNA, 56 bp, artificial sequence
  laboratory synthesized sequence
cccgagctcatctggcataattctcctagttttccagttctccctgcagctgaact

SEQ ID NO: 112 - DNA, 56 bp, artificial sequence
  laboratory synthesized sequence
cccgagctcatctggcataattctcctagttttcgcacggcatatctgcagatctg

SEQ ID NO: 113 - DNA, 53 bp, artificial sequence
  laboratory synthesized sequence
cccgagctcatctggcataattctcctagttttcacctgaggagacggtgacc

SEQ ID NO: 114 - DNA, 56 bp, artificial sequence
  laboratory synthesized sequence
gaaaactaggagaattatgccagatgagctcgggggaccttggtcccagttccgaa

SEQ ID NO: 115 - DNA, 53 bp, artificial sequence
  laboratory synthesized sequence
gaaaactaggagaattatgccagatgagctcggggcttggtccctccgccgaa

SEQ ID NO: 116 - DNA, 53 bp, artificial sequence; identical to SEQ ID NO: 115
  laboratory synthesized sequence

SEQ ID NO: 117 - DNA, 54 bp, artificial sequence
  laboratory synthesized sequence
gaaaactaggagaattatgccagatgagctcggggccttggtgccactgccgaa

SEQ ID NO: 118 - DNA, 54 bp, artificial sequence
  laboratory synthesized sequence
gaaaactaggagaattatgccagatgagctcgggggctgggtgcctcctccgaa

SEQ ID NO: 119 - DNA, 56 bp, artificial sequence
  laboratory synthesized sequence
gaaaactaggagaattatgccagatgagctcggggggctccagtctgaggatgagg

SEQ ID NO: 120 - DNA, 56 bp, artificial sequence
  laboratory synthesized sequence
gaaaactaggagaattatgccagatgagctcggggggctccaggctgaggatgagg

SEQ ID NO: 121 - DNA, 56 bp, artificial sequence; identical to SEQ ID NO: 119
  laboratory synthesized sequence

SEQ ID NO: 122 - DNA, 53 bp, artificial sequence
  laboratory synthesized sequence
gaaaactaggagaattatgccagatgagctcgggctccggtccgaggatgagg

SEQ ID NO: 123 - DNA, 55 bp, artificial sequence
  laboratory synthesized sequence
gaaaactaggagaattatgccagatgagctcggggactccagactggggacgagg

SEQ ID NO: 124 - DNA, 54 bp, artificial sequence
  laboratory synthesized sequence
gaaaactaggagaattatgccagatgagctcggggggctccaggctgaggatga

SEQ ID NO: 125 - DNA, 65 bp, artificial sequence
  laboratory synthesized sequence
gaaaactaggagaattatgccagatgagctcgggtgaccatctctgggctccaggctgaggatga

SEQ ID NO: 126 - DNA, 53 bp, artificial sequence
  laboratory synthesized sequence
gaaaactaggagaattatgccagatgagctcgggggctccaggctgaggacga

SEQ ID NO: 127 - DNA, 54 bp, artificial sequence
  laboratory synthesized sequence
gaaaactaggagaattatgccagatgagctcgggagcgggacccaggctatgga

SEQ ID NO: 128 - DNA, 51 bp, artificial sequence
  laboratory synthesized sequence
gaaaactaggagaattatgccagatgagctcgggcagagcccaagccgggg

SEQ ID NO: 129 - DNA, 54 bp, artificial sequence
  laboratory synthesized sequence
gaaaactaggagaattatgccagatgagctcgggggcccaggtggaggatgaag

SEQ ID NO: 130 - DNA, 54 bp, artificial sequence
  laboratory synthesized sequence
gaaaactaggagaattatgccagatgagctcggggatcgaggctggggatgagg

SEQ ID NO: 131 - DNA, 56 bp, artificial sequence
  laboratory synthesized sequence
gaaaactaggagaattatgccagatgagctcgggtgagtccaggcagaagacgagg

SEQ ID NO: 132 - DNA, 54 bp, artificial sequence
  laboratory synthesized sequence
gaaaactaggagaattatgccagatgagctcggggctcaggcggaagatgaggc

SEQ ID NO: 133 - DNA, 51 bp, artificial sequence
  laboratory synthesized sequence
gaaaactaggagaattatgccagatgagctcggggaagccggggatgaggc

SEQ ID NO: 134 - DNA, 54 bp, artificial sequence
  laboratory synthesized sequence
gaaaactaggagaattatgccagatgagctcgggcagcagggtcctgaccgaag

SEQ ID NO: 135 - DNA, 55 bp, artificial sequence
  laboratory synthesized sequence
gaaaactaggagaattatgccagatgagctcgggcagaagatgaggctgactatt

SEQ ID NO: 136 - DNA, 51 bp, artificial sequence
  laboratory synthesized sequence
gaaaactaggagaattatgccagatgagctcgggcggggcccaggttgagg

SEQ ID NO: 137 - DNA, 60 bp, artificial sequence
  laboratory synthesized sequence
gaaaactaggagaattatgccagatgagctcgggccagtctgacgatgaggctgagtatc

SEQ ID NO: 138 - DNA, 58 bp, artificial sequence
  laboratory synthesized sequence
gaaaactaggagaattatgccagatgagctcgggccatctccaacctccagtttgagg

SEQ ID NO: 139 - DNA, 57 bp, artificial sequence
  laboratory synthesized sequence
gaaaactaggagaattatgccagatgagctcgggcatctccagcctccagtctgagg

SEQ ID NO: 140 - DNA, 53 bp, artificial sequence
  laboratory synthesized sequence
gaaaactaggagaattatgccagatgagctcgggccgggctccagtctgagga

SEQ ID NO: 141 - DNA, 58 bp, artificial sequence
  laboratory synthesized sequence
gaaaactaggagaattatgccagatgagctcgggcatctctgggctccagtctgaaga

SEQ ID NO: 142 - DNA, 57 bp, artificial sequence
  laboratory synthesized sequence
gaaaactaggagaattatgccagatgagctcgggatctctgggctccagtctgagga

SEQ ID NO: 143 - DNA, 54 bp, artificial sequence
  laboratory synthesized sequence
gaaaactaggagaattatgccagatgagctcggggctccagcctgaggatgagg

SEQ ID NO: 144 - DNA, 59 bp, artificial sequence
  laboratory synthesized sequence
gaaaactaggagaattatgccagatgagctcgggccatctctggactgaagactgagga

SEQ ID NO: 145 - DNA, 55 bp, artificial sequence
  laboratory synthesized sequence
gaaaactaggagaattatgccagatgagctcggggtcaggtgtgcagcctgagga

SEQ ID NO: 146 - DNA, 51 bp, artificial sequence
  laboratory synthesized sequence
gaaaactaggagaattatgccagatgagctcgggggtgcgcagcctgagga

SEQ ID NO: 147 - DNA, 52 bp, artificial sequence
  laboratory synthesized sequence
gaaaactaggagaattatgccagatgagctcggggggcccaggcagatgatg

SEQ ID NO: 148 - DNA, 60 bp, artificial sequence
  laboratory synthesized sequence
gaaaactaggagaattatgccagatgagctcgggccaggaagaagatgagagtgactacc

SEQ ID NO: 149 - DNA, 55 bp, artificial sequence
  laboratory synthesized sequence
gaaaactaggagaattatgccagatgagctcggggactccagcctgaggacgagg

SEQ ID NO: 150 - DNA, 21 bp, artificial sequence
  laboratory synthesized sequence
cacaggggcacagactgagga

SEQ ID NO: 151 - DNA, 22 bp, artificial sequence
  laboratory synthesized sequence
catcacaggggcacagactgag

SEQ ID NO: 152 - DNA, 25 bp, artificial sequence
  laboratory synthesized sequence
atccagcctgaagatgaagcaatat

SEQ ID NO: 153 - DNA, 21 bp, artificial sequence
  laboratory synthesized sequence
cagtttggttcctccaccgaa

SEQ ID NO: 154 - DNA, 18 bp, artificial sequence
  laboratory synthesized sequence
cttggttccaccgccgaa

SEQ ID NO: 155 - DNA, 20 bp, artificial sequence
  laboratory synthesized sequence
accttggttccactgccgaa

SEQ ID NO: 156 - DNA, 57 bp, artificial sequence
  laboratory synthesized sequence
cccgagctcatctggcataattctcctagttttcgtgaccgtggtccctgtgcccca

SEQ ID NO: 157 - DNA, 57 bp, artificial sequence
  laboratory synthesized sequence
cccgagctcatctggcataattctcctagttttcgtgagagtggtgccttggcccca

SEQ ID NO: 158 - DNA, 52 bp, artificial sequence
  laboratory synthesized sequence
cccgagctcatctggcataattctcctagttttccagagtcccttggcccca

SEQ ID NO: 159 - DNA, 56 bp, artificial sequence
  laboratory synthesized sequence
cccgagctcatctggcataattctcctagttttctgactgaggttccttgacccca

SEQ ID NO: 160 - DNA, 57 bp, artificial sequence
  laboratory synthesized sequence
cccgagctcatctggcataattctcctagttttcagcacagcctacatgcaactgag

SEQ ID NO: 161 - DNA, 57 bp, artificial sequence
  laboratory synthesized sequence
cccgagctcatctggcataattctcctagttttcagcactgcctacatggagctcag

SEQ ID NO: 162 - DNA, 57 bp, artificial sequence
  laboratory synthesized sequence
cccgagctcatctggcataattctcctagttttcagcacagcctacatgcagctgag

SEQ ID NO: 163 - DNA, 58 bp, artificial sequence
  laboratory synthesized sequence
cccgagctcatctggcataattctcctagttttccaacacagcctacatgcaactcag

SEQ ID NO: 164 - DNA, 57 bp, artificial sequence
  laboratory synthesized sequence
cccgagctcatctggcataattctcctagttttccagcacagtgtacatggtgttga

SEQ ID NO: 165 - DNA, 57 bp, artificial sequence
  laboratory synthesized sequence
cccgagctcatctggcataattctcctagttttcagcacagcctacatgcagctcag

SEQ ID NO: 166 - DNA, 57 bp, artificial sequence
  laboratory synthesized sequence
cccgagctcatctggcataattctcctagttttcagcacagcctacatggagctcag

SEQ ID NO: 167 - DNA, 57 bp, artificial sequence
  laboratory synthesized sequence
cccgagctcatctggcataattctcctagttttcagcacagcctacatggagctccg

SEQ ID NO: 168 - DNA, 54 bp, artificial sequence
  laboratory synthesized sequence
cccgagctcatctggcataattctcctagttttcacagcctatatgcaattcag

SEQ ID NO: 169 - DNA, 56 bp, artificial sequence; identical to SEQ ID NO: 69
  laboratory synthesized sequence

SEQ ID NO: 170 - DNA, 57 bp, artificial sequence
  laboratory synthesized sequence
cccgagctcatctggcataattctcctagttttcagcacagcctacatggagctcaa

SEQ ID NO: 171 - DNA, 52 bp, artificial sequence
  laboratory synthesized sequence
cccgagctcatctggcataattctcctagttttcagcccacatggagctccg

SEQ ID NO: 172 - DNA, 57 bp, artificial sequence; identical to SEQ ID NO: 167
  laboratory synthesized sequence

SEQ ID NO: 173 - DNA, 57 bp, artificial sequence; identical to SEQ ID NO: 167
  laboratory synthesized sequence

SEQ ID NO: 174 - DNA, 57 bp, artificial sequence; identical to SEQ ID NO: 167
  laboratory synthesized sequence

SEQ ID NO: 175 - DNA, 57 bp, artificial sequence; identical to SEQ ID NO: 167
  laboratory synthesized sequence

SEQ ID NO: 176 - DNA, 57 bp, artificial sequence
  laboratory synthesized sequence
cccgagctcatctggcataattctcctagttttcagcacagcctacatggagctaaa

SEQ ID NO: 177 - DNA, 56 bp, artificial sequence
  laboratory synthesized sequence
cccgagctcatctggcataattctcctagttttcacagcccacatggagctcctat

SEQ ID NO: 178 - DNA, 57 bp, artificial sequence
  laboratory synthesized sequence
cccgagctcatctggcataattctcctagttttcagcacagcctacatgcagctcaa

SEQ ID NO: 179 - DNA, 57 bp, artificial sequence; identical to SEQ ID NO: 178
  laboratory synthesized sequence

SEQ ID NO: 180 - DNA, 61 bp, artificial sequence
  laboratory synthesized sequence
cccgagctcatctggcataattctcctagttttcctctagcacagtctacttggagctcag

SEQ ID NO: 181 - DNA, 61 bp, artificial sequence; identical to SEQ ID NO: 180
  laboratory synthesized sequence

SEQ ID NO: 182 - DNA, 57 bp, artificial sequence
  laboratory synthesized sequence
cccgagctcatctggcataattctcctagttttcagcacagcctacatggaactcag

SEQ ID NO: 183 - DNA, 57 bp, artificial sequence; identical to SEQ ID NO: 165
  laboratory synthesized sequence

SEQ ID NO: 184 - DNA, 57 bp, artificial sequence; identical to SEQ ID NO: 165
  laboratory synthesized sequence

SEQ ID NO: 185 - DNA, 57 bp, artificial sequence
  laboratory synthesized sequence
cccgagctcatctggcataattctcctagttttcaagttcaagagcaaggccacact

SEQ ID NO: 186 - DNA, 57 bp, artificial sequence
  laboratory synthesized sequence
cccgagctcatctggcataattctcctagttttcagcactgcctacatgcagctcag

SEQ ID NO: 187 - DNA, 59 bp, artificial sequence
  laboratory synthesized sequence
cccgagctcatctggcataattctcctagttttcagaagttcaagagcaaggccacact

SEQ ID NO: 188 - DNA, 57 bp, artificial sequence; identical to SEQ ID NO: 165
  laboratory synthesized sequence

SEQ ID NO: 189 - DNA, 57 bp, artificial sequence; identical to SEQ ID NO: 165
  laboratory synthesized sequence

SEQ ID NO: 190 - DNA, 58 bp, artificial sequence
  laboratory synthesized sequence
cccgagctcatctggcataattctcctagttttcagcctgacatctgaggactctgcg

SEQ ID NO: 191 - DNA, 57 bp, artificial sequence; identical to SEQ ID NO: 165
  laboratory synthesized sequence

SEQ ID NO: 192 - DNA, 57 bp, artificial sequence
  laboratory synthesized sequence
cccgagctcatctggcataattctcctagttttcgacacatcctccagcacagccta

SEQ ID NO: 193 - DNA, 62 bp, artificial sequence
  laboratory synthesized sequence
cccgagctcatctggcataattctcctagttttccctccagcacagtctatatggagcttag

SEQ ID NO: 194 - DNA, 57 bp, artificial sequence; identical to SEQ ID NO: 165
  laboratory synthesized sequence

SEQ ID NO: 195 - DNA, 57 bp, artificial sequence
  laboratory synthesized sequence
cccgagctcatctggcataattctcctagttttcagcacagcctacatgcagttcag

SEQ ID NO: 196 - DNA, 57 bp, artificial sequence
  laboratory synthesized sequence
cccgagctcatctggcataattctcctagttttcagcacagcctacatgcaactcag

SEQ ID NO: 197 - DNA, 57 bp, artificial sequence; identical to SEQ ID NO: 186
  laboratory synthesized sequence

SEQ ID NO: 198 - DNA, 57 bp, artificial sequence
  laboratory synthesized sequence
cccgagctcatctggcataattctcctagttttcagcacagcctatatggaacttgc

SEQ ID NO: 199 - DNA, 58 bp, artificial sequence
  laboratory synthesized sequence
cccgagctcatctggcataattctcctagttttcaaagttcaagggcaagtccacatt

SEQ ID NO: 200 - DNA, 64 bp, artificial sequence
  laboratory synthesized sequence
cccgagctcatctggcataattctcctagttttcgcacagtctatatggagcttagtagattga

SEQ ID NO: 201 - DNA, 57 bp, artificial sequence; identical to SEQ ID NO: 185
  laboratory synthesized sequence

SEQ ID NO: 202 - DNA, 54 bp, artificial sequence
  laboratory synthesized sequence
cccgagctcatctggcataattctcctagttttcgttcaagggcaaggccacat

SEQ ID NO: 203 - DNA, 57 bp, artificial sequence
  laboratory synthesized sequence
cccgagctcatctggcataattctcctagttttcagcacagcctacatgttgctcag

SEQ ID NO: 204 - DNA, 57 bp, artificial sequence; identical to SEQ ID NO: 165
  laboratory synthesized sequence

SEQ ID NO: 205 - DNA, 57 bp, artificial sequence; identical to SEQ ID NO: 165
  laboratory synthesized sequence

SEQ ID NO: 206 - DNA, 57 bp, artificial sequence; identical to SEQ ID NO: 178
  laboratory synthesized sequence

SEQ ID NO: 207 - DNA, 57 bp, artificial sequence; identical to SEQ ID NO: 165
  laboratory synthesized sequence

SEQ ID NO: 208 - DNA, 54 bp, artificial sequence
  laboratory synthesized sequence
cccgagctcatctggcataattctcctagttttcacagcgtacatggagctccg

SEQ ID NO: 209 - DNA, 57 bp, artificial sequence; identical to SEQ ID NO: 196
  laboratory synthesized sequence

SEQ ID NO: 210 - DNA, 57 bp, artificial sequence; identical to SEQ ID NO: 165
  laboratory synthesized sequence

SEQ ID NO: 211 - DNA, 54 bp, artificial sequence
  laboratory synthesized sequence
cccgagctcatctggcataattctcctagttttcacagcgtacatggagctcca

SEQ ID NO: 212 - DNA, 62 bp, artificial sequence
  laboratory synthesized sequence
cccgagctcatctggcataattctcctagttttcaagagccaagttttctttaaaatgaaca

SEQ ID NO: 213 - DNA, 62 bp, artificial sequence
  laboratory synthesized sequence
cccgagctcatctggcataattctcctagttttcaagagccaagttttcttaaaactgaaca

SEQ ID NO: 214 - DNA, 62 bp, artificial sequence; identical to SEQ ID NO: 212
  laboratory synthesized sequence

SEQ ID NO: 215 - DNA, 62 bp, artificial sequence; identical to SEQ ID NO: 212
  laboratory synthesized sequence

SEQ ID NO: 216 - DNA, 61 bp, artificial sequence
  laboratory synthesized sequence
cccgagctcatctggcataattctcctagttttccaagagccaagttttcttaaaaatgaa

SEQ ID NO: 217 - DNA, 62 bp, artificial sequence
  laboratory synthesized sequence
cccgagctcatctggcataattctcctagttttcaagagccaagttttcttaaaaatgaaca

SEQ ID NO: 218 - DNA, 62 bp, artificial sequence
  laboratory synthesized sequence
cccgagctcatctggcataattctcctagttttcaagagccaagttttctttaaaatgagca

SEQ ID NO: 219 - DNA, 62 bp, artificial sequence
  laboratory synthesized sequence
cccgagctcatctggcataattctcctagttttccaagagccaagttttcttaaaaatgaac

SEQ ID NO: 220 - DNA, 63 bp, artificial sequence
  laboratory synthesized sequence
cccgagctcatctggcataattctcctagttttccaagagtcaagttttcttaaaaatgaaca

SEQ ID NO: 221 - DNA, 60 bp, artificial sequence
  laboratory synthesized sequence
cccgagctcatctggcataattctcctagttttcaagaaccatttcttcctgaagttgaa

SEQ ID NO: 222 - DNA, 60 bp, artificial sequence
  laboratory synthesized sequence
cccgagctcatctggcataattctcctagttttcgaaccagttctccctggaattgaatt

SEQ ID NO: 223 - DNA, 60 bp, artificial sequence
  laboratory synthesized sequence
cccgagctcatctggcataattctcctagttttcgaaccagttctcactgaagttgagtt

SEQ ID NO: 224 - DNA, 61 bp, artificial sequence
  laboratory synthesized sequence
cccgagctcatctggcataattctcctagttttcagaaccagttattcctgcagttgaact

SEQ ID NO: 225 - DNA, 61 bp, artificial sequence
  laboratory synthesized sequence
cccgagctcatctggcataattctcctagttttcagaaccagttcttcctggaaatgaact

SEQ ID NO: 226 - DNA, 61 bp, artificial sequence
  laboratory synthesized sequence
cccgagctcatctggcataattctcctagttttcaaaactcactgtatctgcaaatgaaca

SEQ ID NO: 227 - DNA, 60 bp, artificial sequence
  laboratory synthesized sequence
cccgagctcatctggcataattctcctagttttcaagaaccagtattacctgcagttgaa

SEQ ID NO: 228 - DNA, 60 bp, artificial sequence
  laboratory synthesized sequence
cccgagctcatctggcataattctcctagttttcaaaaatacgctgtacctgcaaatgag

SEQ ID NO: 229 - DNA, 57 bp, artificial sequence
  laboratory synthesized sequence
cccgagctcatctggcataattctcctagttttctacgctgtacctgcaaatgagca

SEQ ID NO: 230 - DNA, 56 bp, artificial sequence
  laboratory synthesized sequence
cccgagctcatctggcataattctcctagttttcaccctgtacctgcaaatgagca

SEQ ID NO: 231 - DNA, 60 bp, artificial sequence
  laboratory synthesized sequence
cccgagctcatctggcataattctcctagttttcaagaacaacctgtacctgcaaatgag

SEQ ID NO: 232 - DNA, 58 bp, artificial sequence
  laboratory synthesized sequence
cccgagctcatctggcataattctcctagttttcgaacaccctgtacctgcaaatgag

SEQ ID NO: 233 - DNA, 58 bp, artificial sequence; identical to SEQ ID NO: 232
  laboratory synthesized sequence

SEQ ID NO: 234 - DNA, 58 bp, artificial sequence; identical to SEQ ID NO: 232
  laboratory synthesized sequence

SEQ ID NO: 235 - DNA, 58 bp, artificial sequence; identical to SEQ ID NO: 232
  laboratory synthesized sequence

SEQ ID NO: 236 - DNA, 59 bp, artificial sequence
  laboratory synthesized sequence
cccgagctcatctggcataattctcctagttttcagaacaccctttacctgcaaatgac

SEQ ID NO: 237 - DNA, 59 bp, artificial sequence
  laboratory synthesized sequence
cccgagctcatctggcataattctcctagttttcagaacaccctgtacctggaaatgag

SEQ ID NO: 238 - DNA, 62 bp, artificial sequence
  laboratory synthesized sequence
cccgagctcatctggcataattctcctagttttccaaagaacattctatacctgcaaatgag

SEQ ID NO: 239 - DNA, 57 bp, artificial sequence
  laboratory synthesized sequence
cccgagctcatctggcataattctcctagttttcaacaccctgttcctgcaaatgac

SEQ ID NO: 240 - DNA, 61 bp, artificial sequence
  laboratory synthesized sequence
cccgagctcatctggcataattctcctagttttcccaaaagtagtgtctacctgcaaatga

SEQ ID NO: 241 - DNA, 64 bp, artificial sequence
  laboratory synthesized sequence
cccgagctcatctggcataattctcctagttttctttcaaaagtagtgtctacctgcatatgaa

SEQ ID NO: 242 - DNA, 61 bp, artificial sequence; identical to SEQ ID NO: 240
  laboratory synthesized sequence

SEQ ID NO: 243 - DNA, 62 bp, artificial sequence
  laboratory synthesized sequence
cccgagctcatctggcataattctcctagttttcccaaaagtagtgtctacctgcaaatgaa

SEQ ID NO: 244 - DNA, 62 bp, artificial sequence
  laboratory synthesized sequence
cccgagctcatctggcataattctcctagttttcgcatcctctatcttcaaatgaacacact

SEQ ID NO: 245 - DNA, 62 bp, artificial sequence
  laboratory synthesized sequence
cccgagctcatctggcataattctcctagttttcgcatcctctaccttcagatgaatgccct

SEQ ID NO: 246 - DNA, 62 bp, artificial sequence; identical to SEQ ID NO: 244
  laboratory synthesized sequence

SEQ ID NO: 247 - DNA, 59 bp, artificial sequence
  laboratory synthesized sequence
cccgagctcatctggcataattctcctagttttccatcctctatcttcaaatgaatgcc

SEQ ID NO: 248 - DNA, 60 bp, artificial sequence
  laboratory synthesized sequence
cccgagctcatctggcataattctcctagttttcatcctctatcttcaaatgaacaccct

SEQ ID NO: 249 - DNA, 62 bp, artificial sequence
  laboratory synthesized sequence
cccgagctcatctggcataattctcctagttttcaccaagtattccttaaactcaccagtgt

SEQ ID NO: 250 - DNA, 58 bp, artificial sequence
  laboratory synthesized sequence
cccgagctcatctggcataattctcctagttttccaacaaccaagcattcctgaatat

SEQ ID NO: 251 - DNA, 61 bp, artificial sequence
  laboratory synthesized sequence
cccgagctcatctggcataattctcctagttttcaaccaggtattcctcaagatcaccagt

SEQ ID NO: 252 - DNA, 61 bp, artificial sequence
  laboratory synthesized sequence
cccgagctcatctggcataattctcctagttttcaaccaggtattcctcaagatcaccact

SEQ ID NO: 253 - DNA, 57 bp, artificial sequence
  laboratory synthesized sequence
cccgagctcatctggcataattctcctagttttcaaccaggtattcctcaagatcgc

SEQ ID NO: 254 - DNA, 57 bp, artificial sequence
  laboratory synthesized sequence
cccgagctcatctggcataattctcctagttttcaggtattcctcaagatcgccagt

SEQ ID NO: 255 - DNA, 59 bp, artificial sequence
  laboratory synthesized sequence
cccgagctcatctggcataattctcctagttttcccaggtattcctcaagatcaccagt

SEQ ID NO: 256 - DNA, 57 bp, artificial sequence
  laboratory synthesized sequence
cccgagctcatctggcataattctcctagttttcagcactgcctatttgcagatcaa

SEQ ID NO: 257 - DNA, 57 bp, artificial sequence; identical to SEQ ID NO: 256
  laboratory synthesized sequence

SEQ ID NO: 258 - DNA, 57 bp, artificial sequence; identical to SEQ ID NO: 256
  laboratory synthesized sequence

SEQ ID NO: 259 - DNA, 60 bp, artificial sequence
  laboratory synthesized sequence
cccgagctcatctggcataattctcctagttttcgccagcactgcctatttacagataag

SEQ ID NO: 260 - DNA, 60 bp, artificial sequence
  laboratory synthesized sequence
cccgagctcatctggcataattctcctagttttcgaaagcatgctctatctgcaaatgaa

SEQ ID NO: 261 - DNA, 54 bp, artificial sequence
  laboratory synthesized sequence
cccgagctcatctggcataattctcctagttttcgctctatctgcaaatgaaca

SEQ ID NO: 262 - DNA, 56 bp, artificial sequence
  laboratory synthesized sequence
cccgagctcatctggcataattctcctagttttcaccctgtacctgcagatgagca

SEQ ID NO: 263 - DNA, 56 bp, artificial sequence; identical to SEQ ID NO: 262
  laboratory synthesized sequence

SEQ ID NO: 264 - DNA, 59 bp, artificial sequence
  laboratory synthesized sequence
cccgagctcatctggcataattctcctagttttcagaaccagttcttcctccaattgaa

SEQ ID NO: 265 - DNA, 57 bp, artificial sequence
  laboratory synthesized sequence
cccgagctcatctggcataattctcctagttttcagcagtgcatacatgcagatgaa

SEQ ID NO: 266 - DNA, 63 bp, artificial sequence
  laboratory synthesized sequence
cccgagctcatctggcataattctcctagttttctcaaaaagcagtgtctacctagagatgaa

SEQ ID NO: 267 - DNA, 55 bp, artificial sequence
  laboratory synthesized sequence
cccgagctcatctggcataattctcctagttttccacagcctacctgcagctcag

SEQ ID NO: 268 - DNA, 55 bp, artificial sequence; identical to SEQ ID NO: 267
  laboratory synthesized sequence

SEQ ID NO: 269 - DNA, 55 bp, artificial sequence; identical to SEQ ID NO: 267
  laboratory synthesized sequence

SEQ ID NO: 270 - DNA, 55 bp, artificial sequence; identical to SEQ ID NO: 267
  laboratory synthesized sequence

SEQ ID NO: 271 - DNA, 58 bp, artificial sequence
  laboratory synthesized sequence
cccgagctcatctggcataattctcctagttttccaacacagcctacttggagctcaa

SEQ ID NO: 272 - DNA, 66 bp, artificial sequence
  laboratory synthesized sequence
cccgagctcatctggcataattctcctagttttcttctgagaacttattgtatctacaaatgaaca

SEQ ID NO: 273 - DNA, 55 bp, artificial sequence
  laboratory synthesized sequence
gaaaactaggagaattatgccagatgagctcgggcagtttggttcctccaccgaa

SEQ ID NO: 274 - DNA, 52 bp, artificial sequence
  laboratory synthesized sequence
gaaaactaggagaattatgccagatgagctcgggcttggttccaccgccgaa

SEQ ID NO: 275 - DNA, 54 bp, artificial sequence
  laboratory synthesized sequence
gaaaactaggagaattatgccagatgagctcgggaccttggttccactgccgaa

SEQ ID NO: 276 - DNA, 55 bp, artificial sequence
  laboratory synthesized sequence
gaaaactaggagaattatgccagatgagctcgggcacaggggcacagactgagga

SEQ ID NO: 277 - DNA, 56 bp, artificial sequence
  laboratory synthesized sequence
gaaaactaggagaattatgccagatgagctcgggcatcacaggggcacagactgag

SEQ ID NO: 278 - DNA, 59 bp, artificial sequence
  laboratory synthesized sequence
gaaaactaggagaattatgccagatgagctcgggatccagcctgaagatgaagcaatat

(19) United States
(12) Patent Application Publication
(10) Pub. No.: US 2015/0167078 A1
(43) Pub. Date: Jun. 18, 2015

(54) METHODS FOR ANTIBODY AND CELL THERAPEUTIC DISCOVERY

(71) Applicant: Gigagen, Inc., San Francisco, CA (US)

(72) Inventors: David Scott Johnson, San Francisco, CA (US); Andrea Loehr, San Francisco, CA (US)

(21) Appl. No.: 14/568,377

(22) Filed: Dec. 12, 2014

Related U.S. Application Data
(63) Continuation of application No. PCT/US2013/045904, filed on Jun. 14, 2013.
(60) Provisional application No. 61/659,791, filed on Jun. 14, 2012.
(60) Provisional application No. 61/662,538, filed on Jun. 21, 2012.

(51) Int. Cl. C12Q 1/68

(52) U.S. Cl. CPC C12Q 1/6881; C12Q 2600/16; C12Q 2600/112

(57) ABSTRACT

A method is provided for discovering antibodies with desired properties. In one aspect of this invention, oligoclonal single chain repertoires are determined in a target tissue. Single cells from blood are then isolated and sequenced to determine the heavy and light chain pairing in each of thousands of cells. The oligoclonal single chain repertoire is then used to computationally select candidate paired chain antibodies to which the target tissue is antigenic. In another aspect of the invention, oligoclonal heavy and light chain repertoires are determined separately in a target tissue. A list of candidate fully paired antibodies are then identified computationally by an all-by-all pairing of the oligoclonal heavy chain repertoire with the oligoclonal light chain repertoire. This invention has practical application in many biological and medical specialties where antibodies are useful, such as therapeutics, diagnostics, molecular analysis, oncology, transplantation, and infectious disease.

### CROSS REFERENCE TO RELATED APPLICATIONS

This application claims the benefit of U.S. Provisional Application No. 61/662,538 filed Jun. 21, 2012 and U.S. Provisional Application No. 61/659,791, filed Jun. 14, 2012 both entitled “Methods for Antibody and Cell Therapeutic Discovery” which are hereby incorporated by reference in their entireties.

### 1. FIELD OF THE INVENTION

The invention relates to the field of functional antibody and T cell receptor discovery, specifically to the use of endogenous immune repertoires to discover antibodies (including native human antibodies) or T cell receptors with desired antigenic properties.

### 2. BACKGROUND OF THE INVENTION

### 2.1. Introduction

Antibody therapeutics are increasingly used by pharmaceutical companies to treat intractable diseases such as cancer (Carter 2006 Nature Reviews Immunology 6:343-357; Carter 2011 Experimental Cell Res 317 1261-1269; Reichart 2011 mAbs 3:1 76-99; Reichart 2010 mAbs 2:6 695-700). However, the current process of antibody drug discovery is expensive and tedious, requiring the identification of an antigen, and then the isolation and production of monoclonal antibodies with activity against the antigen. Individuals that have been exposed to disease produce a wide variety of antibodies to protect them from disease. Only a small fraction of these native antibodies are specific to discrete, isolated, identifiable antigens.

These immune repertoires contain antibodies with high affinity against potential therapeutic or analytical targets and which are also preselected for low immunogenicity. In general, antibodies with native heavy and light Ig pairing have a higher probability of utility in a practical and clinical context.

### 3. SUMMARY OF THE INVENTION

In particular non-limiting embodiments, the invention is directed to a method for discovering antibodies, comprising: (a) identifying single chain immunoglobulin sequences in a target tissue from an animal; (b) providing a first set of nucleic acid probes, (i) the first set comprising a first probe comprising a sequence that is complementary to an immunoglobulin heavy chain sequence, (ii) a second probe comprising a sequence that is complementary to the immunoglobulin heavy chain sequence and a second sequence that is complementary to an exogenous sequence, (iii) a third probe comprising a sequence that is complementary to the portion of the second probe that is complementary to the exogenous sequence and a sequence that is complementary to an immunoglobulin light chain sequence, and (iv) a fourth probe comprising a sequence that is complementary to an immunoglobulin light chain sequence; (c) isolating single antibody-producing cells from said animal with at least one set of said nucleic acid probes; (d) amplifying immunoglobulin heavy and light chain targets independently, wherein the heavy chain sequence is amplified using the first probe and the second probe, and wherein the light chain sequence is amplified using the third probe and the fourth probe; (e) generating a fused complex by hybridizing the complementary sequence regions of the amplified said heavy and light chain sequences and amplifying the hybridized sequences using the first and fourth probes; (f) performing a bulk sequencing reaction to generate sequence information for at least 100,000 fused complexes from at least 10,000 cells within the population of cells, wherein the sequence information is sufficient to co-localize the first target nucleic acid sequence and the second target nucleic acid sequence to a single cell from the population of at least 10,000 cells; and (g) computationally matching said oligoclonal single chain immunoglobulin sequences with single chains from said fused complexes to identify a candidate list of paired immunoglobulin sequences to which the target tissue may be antigenic.

In other non-limiting embodiments, the invention is directed to a method for discovering antibodies, comprising: (a) identifying heavy chain immunoglobulin sequences in a target tissue from an animal; (b) identifying light chain immunoglobulin sequences in said target tissue from said animal; and (c) computationally pairing said identified single chain immunoglobulin sequences more frequent than 1% of clones with each said identified light chain immunoglobulin sequences more frequent than 1% of clones to obtain a candidate list of paired immunoglobulin sequences to which the target tissue may be antigenic.

In additional non-limiting embodiments, the invention is directed to a method for discovering antibodies, comprising: (a) identifying heavy and light chain immunoglobulin sequences in a target tissue from an animal with a disease or condition; (b) identifying heavy and light chain immunoglobulin sequences in said target tissue from an animal without said disease or condition; (c) obtaining a list of single heavy and light chain immunoglobulin sequences more frequent than 1% of clones in the animal with said disease or condition but not more frequent than 1% of clones in the animal without said disease or condition; and (d) computationally pairing heavy and light chain to obtain a candidate list of paired immunoglobulin sequences that may be associated with said disease or condition.

For the invention above, the number of animals surveyed may be 1, 10, 100, 1000, or 10000.

In the methods above, the cells may be B cells, bone marrow plasma cells, or plasma cells. The animal may be avian, mammalian or reptilian. If a mammal it may be a camel, a human, a mouse, a rabbit, a rat, or a sheep.

The tissue may be a tissue sampled from an animal with a disease selected from the group consisting of: multiple sclerosis, rheumatoid arthritis, ulcerative colitis, Crohn's disease, systemic lupus erythematosus, graft-versus-host-disease, and host-versus-graft disease. The tissue may also be tumor tissue selected from the group consisting of: lung carcinoma, non-small cell lung cancer, small cell lung cancer, uterine cancer, thyroid cancer, breast carcinoma, prostate carcinoma, pancreas carcinoma, colon carcinoma, lymphoma, Burkitt lymphoma, Hodgkin lymphoma, myeloid leukemia, leukemia, sarcoma, blastoma, melanoma, seminoma, brain cancer, glioma, glioblastoma, cerebellar astrocytoma, cutaneous T-cell lymphoma, gastric cancer, liver cancer, ependymona, laryngeal cancer, neck cancer, stomach cancer, kidney cancer, pancreatic cancer, bladder cancer, esophageal cancer, testicular cancer, medulloblastoma, vaginal cancer, ovarian cancer, cervical cancer, basal cell carcinoma, pituitary adenoma, rhabdomyosarcoma, and Kaposi sarcoma.

### 4. BRIEF DESCRIPTION OF THE FIGURES

FIG. 1. Schematic showing the plasmids involved in the cloning of the antibodies.

FIG. 2. Four primers and two targets required to initiate an overlap extension PCR.

FIG. 3. Primer pairs independently amplify each minor amplicon.

### 5. DETAILED DESCRIPTION OF THE INVENTION

### 5.1. Definitions

Terms used in the claims and specification are defined as set forth below unless otherwise specified.

The term “antibody” refers to an intact immunoglobulin of any isotype, or a fragment thereof that can compete with the intact antibody for specific binding to the target antigen, and includes chimeric, humanized, fully human, and bispecific antibodies. An intact antibody generally will comprise at least two full-length heavy chains and two full-length light chains, but in some instances may include fewer chains such as antibodies naturally occurring in camelids which may comprise only heavy chains. Antibodies according to the invention may be derived solely from a single source, or may be “chimeric,” that is, different portions of the antibody may be derived from two different antibodies. For example, the CDR regions may be derived from a rat or murine source, while the framework region of the V region are derived from a different animal source, such as a human. The antibodies or binding fragments of the invention may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below.

The term “B cell” refers to a type of lymphocyte that plays a large role in the humoral immune response (as opposed to the cell-mediated immune response, which is governed by T cells). The principal functions of B cells are to make antibodies against antigens, perform the role of antigen-presenting cells (APCs) and eventually develop into memory B cells after activation by antigen interaction. B cells are an essential component of the adaptive immune system.

A “bispecific,” “dual-specific” or “bifunctional” antibody is a hybrid antibody having two different antigen binding sites. Bispecific antibodies are a species of multispecific antibody and may be produced by a variety of methods including, but not limited to, fusion of hybridomas or linking of Fab′ fragments. See, e.g., Songsivilai & Lachmann (1990), Clin. Exp. Immunol. 79:315-321; Kostelny et al. (1992), J. Immunol. 148: 1547-1553. The two binding sites of a bispecific antibody will bind to two different epitopes, which may reside on the same or different protein targets.

A “bivalent antibody” comprises two antigen binding sites. In some instances, the two binding sites have the same antigen specificities. However, bivalent antibodies may be bispecific (see below).

The term “bulk sequencing” or “next generation sequencing” or “massively parallel sequencing” refers to any high throughput sequencing technology that parallelizes the DNA sequencing process. For example, bulk sequencing methods are typically capable of producing more than one million polynucleic acid amplicons in a single assay. The terms “bulk sequencing,” “massively parallel sequencing,” and “next generation sequencing” refer only to general methods, not necessarily to the acquisition of greater than 1 million sequence sequences in a single run. Any bulk sequencing method can be implemented in the invention, such as reversible terminator chemistry (e.g., Illumina), pyrosequencing using polony emulsion droplets (e.g., Roche), ion semiconductor sequencing (IonTorrent), single molecule sequencing (e.g., Pacific Biosciences), massively parallel signature sequencing, etc.

The term “cell” refers to a functional basic unit of living organisms. A cell includes any kind of cell (prokaryotic or eukaryotic) from a living organism. Examples include, but are not limited to, mammalian mononuclear blood cells, yeast cells, or bacterial cells.

The term “cell therapy” refers to the medical practice of using cell grafts as therapy for disease. In certain embodiments, cell therapy involves in vitro expansion of T cell cells directed against a particular antigen, or artificially engineered to produce a T cell receptor with therapeutic benefit. Usually cell therapy takes advantage of cells expressing genes with variable regions, such as B cells or T cells.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VII regions of a bivalent domain antibody may target the same or different antigens.

“Droplet” refers to a small volume of liquid, typically with a spherical shape or as a slug that fills the diameter of a microchannel, encapsulated by an immiscible fluid. The volume of a droplet, and/or the average volume of droplets in an emulsion, may be less than about one microliter (i.e., a “microdroplet”) (or between about one microliter and one nanoliter or between about one microliter and one picoliter), less than about one nanoliter (or between about one nanoliter and one picoliter), or less than about one picoliter (or between about one picoliter and one femtoliter), among others. A droplet may have a diameter (or an average diameter) of less than about 1000, 100, or 10 micrometers, or of about 1000 to 10 micrometers, among others. A droplet may be spherical or nonspherical. In some embodiments, the droplet has a volume and diameter that is large enough to encapsulate a cell.

The term “emulsion droplet” or “emulsion microdroplet” refers to a droplet that is formed when two immiscible fluids are combined. For example, an aqueous droplet can be formed when an aqueous fluid is mixed with a non-aqueous fluid. In another example, a nonaqueous fluid can be added to an aqueous fluid to form a droplet. Droplets can be formed by various methods, including methods performed by microfluidics devices or other methods, such as injecting one fluid into another fluid, pushing or pulling liquids through an orifice or opening, forming droplets by shear force, etc. The droplets of an emulsion may have any uniform or non-uniform distribution. Any of the emulsions disclosed herein may be monodisperse (composed of droplets of at least generally uniform size), or may be polydisperse (composed of droplets of various sizes). If monodisperse, the droplets of the emulsion may vary in volume by a standard deviation that is less than about plus or minus 100%, 50%, 20%, 10%, 5%, 2%, or 1% of the average droplet volume. Droplets generated from an orifice may be monodisperse or polydisperse. An emulsion may have any suitable composition. The emulsion may be characterized by the predominant liquid compound or type of liquid compound that is used. The predominant liquid compounds in the emulsion may be water and oil. “Oil” is any liquid compound or mixture of liquid compounds that is immiscible with water and that has a high content of carbon. In some examples, oil also may have a high content of hydrogen, fluorine, silicon, oxygen, or any combination thereof, among others. For example, any of the emulsions disclosed herein may be a water-in-oil (W/O) emulsion (i.e., aqueous droplets in a continuous oil phase). The oil may be or include at least one silicone oil, mineral oil, fluorocarbon oil, vegetable oil, or a combination thereof, among others. Any other suitable components may be present in any of the emulsion phases, such as at least one surfactant, reagent, sample (i.e., partitions thereof), buffer, salt, ionic element, other additive, label, particles, or any combination thereof.

A “Fab fragment” is comprised of one light chain and the CHI and variable regions of one heavy chain. The heavy chain of a Fab molecule cannot form a disulfide bond with another heavy chain molecule.

A “Fab′ fragment” contains one light chain and a portion of one heavy chain that contains the VH domain and the CHI domain and also the region between the CHI and CH2 domains, such that an interchain disulfide bond can be formed between the two heavy chains of two Fab′ fragments to form a F(ab′)2 molecule.

A “F(ab′)2 fragment” contains two light chains and two heavy chains containing a portion of the constant region between the CHI and CH2 domains, such that an interchain disulfide bond is formed between the two heavy chains. A F(ab′)2 fragment thus is composed of two Fab′ fragments that are held together by a disulfide bond between the two heavy chains.

An “Fc” region contains two heavy chain fragments comprising the CH2 and CH3 domains of an antibody and in some cases the lower hinge region. The two heavy chain fragments are held together by two or more disulfide bonds (typically in the hinge region) and by hydrophobic interactions of the CH3 domains.

The “Fv region” comprises the variable regions from both the heavy and light chains, but lacks the constant regions.

The term “gene” refers to a nucleic acid sequence that can be potentially transcribed and/or translated which may include the regulatory elements in 5′ and 3′, and the introns, if present.

The term “heavy chain” includes a full-length heavy chain and fragments thereof having sufficient variable region sequence to confer binding specificity. A full-length heavy chain includes a variable region domain, VH, and three constant region domains, CHI, CH2, and CH3. The VH domain is at the amino-terminus of the polypeptide, and the CH domains are at the carboxyl-terminus, with the CH3 being closest to the —COOH end. Heavy chains according to the invention may be of any isotype, including IgG (including IgG1, IgG2, IgG3 and IgG4 subtypes), IgA (including IgA1 and IgA2 subtypes), IgM and IgE.

The term “immunologically functional fragment” (or simply “fragment”) of an immunoglobulin chain, as used herein, refers to a portion of an antibody light chain or heavy chain that lacks at least some of the amino acids present in a full-length chain but which is capable of binding specifically to an antigen. Such fragments are biologically active in that they bind specifically to the target antigen and can compete with intact antibodies for specific binding to a given epitope. In one aspect of the invention, such a fragment will retain at least one CDR present in the full-length light or heavy chain, and in some embodiments will comprise a single heavy chain and/or light chain or portion thereof. These biologically active fragments may be produced by recombinant DNA techniques, or may be produced by enzymatic or chemical cleavage of intact antibodies Immunologically functional immunoglobulin fragments of the invention include, but are not limited to, Fab, Fab′, F(ab′)2, Fv, domain antibodies and single-chain antibodies, and may be derived from any mammalian source, including but not limited to human, mouse, rat, camelid or rabbit. It is contemplated further that a functional portion of the inventive antibodies, for example, one or more CDRs, could be covalently bound to a second protein or to a small molecule to create a therapeutic agent directed to a particular target in the body, possessing bifunctional therapeutic properties, or having a prolonged serum half-life.

The term “ligase chain reaction” or LCR refers to a type of DNA amplification where two DNA probes are ligated by a DNA ligase, and a DNA polymerase is used to amplify the resulting ligation product. Traditional PCR methods are used to amplify the ligated DNA sequence.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains according to the invention include kappa chains and lambda chains.

The term “mammal” as used herein includes both humans and non-humans and include, but is not limited to, humans, non-human primates, canines, felines, murines, bovines, equines, and porcines.

The term “minor amplicon” refers to amplicons that are generated by amplifying the two distinct nucleic acid target sequences separately. The term “major amplicon” refers to a fusion product between at least two minor amplicons as a result of overlap extension PCR. and then fused by amplification to create a fusion amplicon, also known as a “major” amplicon. Sometimes the major amplicon may also be called the “fusion amplicon”, “fusion product”, “linkage product”, or “linkage amplicon”.

A “multispecific antibody” is one that targets more than one antigen or epitope.

The term “neutralizing antibody” refers to an antibody that binds to a ligand, prevents binding of the ligand to its binding partner and interrupts the biological response that otherwise would result from the ligand binding to its binding partner. In assessing the binding and specificity of an antibody or immunologically functional fragment thereof, an antibody or fragment will substantially inhibit binding of a ligand to its binding partner when an excess of antibody reduces the quantity of binding partner bound to the ligand by at least about 20%, 30%, 40%, 50%, 60%, 70%, 80%, 85%, 90%, 95%, 97%, 99% or more (as measured in an in vitro competitive binding assay).

The term “polymerase chain reaction” or PCR refers to a molecular biology technique for amplifying a DNA sequence from a single copy to several orders of magnitude (thousands to millions of copies). PCR relies on thermal cycling, which requires cycles of repeated heating and cooling of the reaction for DNA melting and enzymatic replication of the DNA. Primers (short DNA fragments, or oligonucleotides) containing sequences complementary to the target region of the DNA sequence and a DNA polymerase are key components to enable selective and repeated amplification. As PCR progresses, the DNA generated is itself used as a template for replication, setting in motion a chain reaction in which the DNA template is exponentially amplified. A heat-stable DNA polymerase, such as Taq polymerase, is used. The thermal cycling steps are necessary first to physically separate the two strands in a DNA double helix at a high temperature in a process called DNA melting. At a lower temperature, each strand is then used as the template in DNA synthesis by the DNA polymerase to selectively amplify the target DNA. The selectivity of PCR results from the use of primers that are complementary to the DNA region targeted for amplification under specific thermal cycling conditions.

The term “reverse transcriptase polymerase chain reaction” or RT-PCR refers to a type of PCR reaction used to generate multiple copies of a DNA sequence. In RT-PCR, an RNA strand is first reverse transcribed into its DNA complement (complementary DNA or cDNA) using the enzyme reverse transcriptase, and the resulting cDNA is amplified using traditional PCR techniques.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203.

The term “T cell” refers to a type of cell that plays a central role in cell-mediated immune response. T cells belong to a group of white blood cells known as lymphocytes and can be distinguished from other lymphocytes, such as B cells and natural killer T (NKT) cells by the presence of a T cell receptor (TCR) on the cell surface. T cells responses are antigen specific and are activated by foreign antigens. T cells are activated to proliferate and differentiate into effector cells when the foreign antigen is displayed on the surface of the antigen-presenting cells in peripheral lymphoid organs. T cells recognize fragments of protein antigens that have been partly degraded inside the antigen-presenting cell. There are two main classes of T cells—cytotoxic T cells and helper T cells. Effector cytotoxic T cells directly kill cells that are infected with a virus or some other intracellular pathogen. Effector helper T cells help to stimulate the responses of other cells, mainly macrophages, B cells and cytotoxic T cells.

It must be noted that, as used in the specification and the appended claims, the singular forms “a,” “an,” and “the” include plural referents unless the context clearly dictates otherwise. The present invention may suitably comprise, consist of, or consist essentially of, the steps described in the claims.

### 5.2. General Methods

### 5.2.1. B Cell Analysis and Drug Discovery

Antibody therapeutics are increasingly used by pharmaceutical companies to treat intractable diseases such as cancer (Carter 2006 Nature Reviews Immunology 6:343-357). Methods such as phage display, mammalian cell display, mouse hybridomas, and human B cell immortalization are all used to discover functional antibodies using Ig repertoires (Beerli and Rader, 2010, mAbs 2: 365-378). However, the process of antibody drug discovery remains expensive and tedious, requiring the identification of an antigen, and then the isolation and production of monoclonal antibodies with activity against the antigen. No current conventional screening method can obtain functional natural human antibodies with native Ig heavy and light chain pairings intact. Moreover, many antibodies discovered using current technologies have high immunogenicity when introduced to humans (Suntharalingam et al., 2006, New England Journal of Medicine 355: 1018-28). Individuals that have been exposed to disease produce antibodies against antigens associated with that disease. Thus, it is possible mine patient immune repertoires for specific antibodies that could be used for pharmaceutical development. These antibodies may be used for a variety of uses, e.g., therapeutics, immunohistochemistry (IHC). In one embodiment, antibodies are found by matching the immune repertoire found in a diseased tissue, e.g., tumor infiltrating B lymphocytes, with those found peripheral blood mononuclear cells (PBMCs).

### 5.2.2. T Cell Analysis and Therapeutic Discovery

Recently cell therapy has been used to treat chronic lymphoid leukemia (Porter et al., 2011 New England Journal of Medicine 365:725-733). However, the process of functional T cell discovery is expensive and tedious, requiring the identification of an antigen, and then the isolation and production of T cells with activity against the antigen. No current conventional screening method can obtain functional natural human antibodies with native TCR alpha and beta pairings intact. Individuals that have been exposed to disease produce T cells against antigens associated with that disease. Thus, it is possible mine patient T cell repertoires for specific T cell receptor sequences that could be used for cell therapeutic development. In one embodiment, therapeutic T cells are found by matching the immune repertoire found in a diseased tissue, e.g., tumor infiltrating T lymphocytes, with those found peripheral blood mononuclear cells (PBMCs).

### 5.3. Antibodies

Another aspect of the invention pertains to the development of antibodies or immunologically functional fragments for use as diagnostics or therapeutics discovered by the methods of the invention. Examples of immunologically functional fragments include F(ab) and F(ab′)₂ fragments which can be generated by treating the antibody with an enzyme such as pepsin. Alternatively, immunologically functional fragments may be monomeric binders such as scFv, diabodies, minibodies, small immunoproteins (SIPs). Olafsen et al. 2005 Cancer Res 65:5907-5916; Borsi et al. 2002 Int J Cancer 102:75-85; Berndorff et al. 2005 Clin Cancer Res 11:7053s-7063s; and Tijink et al. 2006 J Nucl Med 47:1127-1135.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies may be prepared by standard methods. Antibody-producing cells can be obtained from the subject and used to prepare monoclonal antibodies by standard techniques, such as the hybridoma technique originally described by Kohler and Milstein 1975 Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Ilybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409 (Winter); PCT Publication Nos. WO 92/18619; WO 91/17271; WO 92/20791; WO 92/15679; WO 93/01288; WO 92/01047; WO 92/09690; WO 90/02809; Fuchs et al. 1991 Bio/Technology 9:1370-1372; Hay et al. 1992 Hum. Antibod. Hybridomas 3:81-85; Huse et al. 1989 Science 246:1215-1281; Griffiths et al. 1993 EMBO J. 12:725-734.

Additionally, recombinant antibodies, such as chimeric and humanized monoclonal antibodies, comprising both human and non-human portions can be made using standard recombinant DNA techniques. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 5,225,539 (Winter); U.S. Pat. No. 4,816,567 (Cabilly et al.); European Patent Application 125,023; Better et al. 1988 Science 240:1041-1043; Liu et al. 1987 Proc. Natl. Acad. Sci. USA 84:3439-3443; Liu et al. 1987 J. Immunol. 139:3521-3526; Sun et al. 1987 Proc. Natl. Acad. Sci. USA 84:214-218; Nishimura et al. 1987 Cancer Res. 47:999-1005; Wood et al. 1985 Nature 314:446-449; and Shaw et al. 1988 J. Natl. Cancer Inst. 80:1553-1559; Morrison 1985 Science 229:1202-1207; Oi et al. 1986 Bio/Techniques 4:214; Jones et al. (1986) Nature 321:552-525; Verhoeyen et al. 1988 Science 239:1534-1536; and Beidler et al. 1988 J. Immunol. 141:4053-4060.

Completely human antibodies can be produced using transgenic mice which are incapable of expressing endogenous immunoglobulin heavy and light chains genes, but which can express human heavy and light chain genes. For an overview of this technology for producing human antibodies, see Lonberg and Iluszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806 (Lonberg et al.). In addition, companies such as Ahgenix, Inc. (Freemont, Calif.), can be engaged to provide human antibodies directed against a selected antigen using technology similar to that described above.

An antibody may be used to detect a marker (e.g., in a cellular lysate or cell supernatant) in order to evaluate the level and pattern of expression of the marker. The antibodies can also be used diagnostically to monitor protein levels in tissues or body fluids (e.g., in a tumor cell-containing body fluid) as part of a clinical testing procedure, e.g., to for example, determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin, and examples of suitable radioactive materials for diagnostics or therapeutics include, but are not limited to, ³H, ¹²⁵1, ¹³¹I, ¹¹¹In, ¹⁷⁷Lu, ⁹⁰Y, or ³⁵S.

The invention also includes therapeutic antibody conjugates. Therapeutic antibody conjugates are well-known in the art and are the basis for a number of approved drugs such as Gemtuzumab ozogamicin (Mylotarg; Wyeth) with calicheamicin toxin; Brentuximab vedotin (Adcetris; Seattle Genetics) with an auristatin toxin; ⁹⁰Y-labelled ibritumomab tiuxetan (Zevalin; IDEC Pharmaceuticals); and ¹³¹I-labelled tositumomab (Bexxar; GlaxoSmithKline), see Scott et al. 2012 Nat Rev Canc 12 278-287. Radioactive conjugates may be high energy β-emitters such as ⁹⁰Y, medium energy β-emitters such as ¹³¹I or ¹⁷⁷Lu, Auger electron emitters such as ¹²⁵I, or ¹¹¹In, or α-emitters such as ²²⁵Ac, ²¹¹At, ²¹³Bi, or ²²⁷Th. Steiner and Neri 2011 Clin Canc Res 17(20) 6406-6416. In addition to the antibody drug conjugates (ADCs) mentioned above, various toxins that target microtubules, DNA or DNA enzymes such as topoisomerase II are in development. Examples of other conjugates in clinical development are dolastatin, doxorubicin, maytansine, or paclitaxel. The linker technology for ADCs is well understood. See for example Iyer and Kadambi 2011 J Pharmacol Tox Meth 64 207-211.

### 5.4. Pharmaceutically Acceptable Compositions

Provided herein are pharmaceutical compositions comprising an antibody as an active ingredient, or a pharmaceutically acceptable salt, solvate or hydrate thereof in combination with a pharmaceutically acceptable vehicle, carrier, diluent, or excipient, or a mixture thereof.

The compound provided herein may be administered alone, or in combination with one or more other compounds provided herein. The pharmaceutical compositions that comprise an antibody can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, 21st Ed., Lippincott, Williams & Wilkins, Baltimore, M.D., 2006; Modified-Release Drug Delivery Technology, Rathbone et al., Eds., Drugs and the Pharmaceutical Science, Marcel Dekker, Inc.: New York, N.Y., 2003; Vol. 126).

In one embodiment, the pharmaceutical compositions are provided in a dosage form for parenteral administration, which comprise an antibody or a pharmaceutically acceptable salt, solvate or hydrate thereof; and one or more pharmaceutically acceptable excipients or carriers.

In yet another embodiment, the pharmaceutical compositions are provided in a dosage form for topical administration, which comprise antibody or a pharmaceutically acceptable salt, solvate or hydrate thereof; and one or more pharmaceutically acceptable excipients or carriers.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons. The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In one embodiment, the therapeutically effective dose is from about 0.1 mg to about 2,000 mg per day of a compound provided herein. The pharmaceutical compositions therefore should provide a dosage of from about 0.1 mg to about 2000 mg of the compound. In certain embodiments, pharmaceutical dosage unit forms are prepared to provide from about 1 mg to about 2000 mg, from about 10 mg to about 1000 mg, from about 20 mg to about 500 mg or from about 25 mg to about 250 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

### 5.4.1. Parental Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carhoxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein can be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In one embodiment, the lyophilized nanoparticles are provided in a vial for reconstitution with a sterile aqueous solution just prior to injection. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions. The pharmaceutical compositions provided herein can be formulated as immediate or modified release dosage forms, including delayed-, sustained, pulsed-, controlled, targeted-, and programmed-release forms.

The pharmaceutical compositions can be formulated as a suspension, solid, semi-solid, or thixotropic liquid, for administration as an implanted depot.

### 5.5. Dosage

The pharmaceutical compositions that are provided can be administered for prophylactic and/or therapeutic treatments. An “effective amount” refers generally to an amount that is a sufficient, but non-toxic, amount of the active ingredient (i.e., antibody) to achieve the desired effect, which is a reduction or elimination in the severity and/or frequency of symptoms and/or improvement or remediation of damage. A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom.

In general, toxicity and therapeutic efficacy of the antibody can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

The data obtained from cell culture and/or animal studies can be used in formulating a range of dosages for humans. The dosage of the active ingredient typically lines within a range of circulating concentrations that include the ED50 with little or no toxicity. The dosage can vary within this range depending upon the dosage form employed and the route of administration utilized.

The effective amount of a pharmaceutical composition comprising the antibody to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the antibody is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 150 mg/kg; or 1 μg/kg up to about 100 mg/kg; or 5 μg/kg up to about 50 mg/kg.

The dosing frequency will depend upon the pharmacokinetic parameters of the antibody in the formulation. For example, a clinician will administer the composition until a dosage is reached that achieves the desired effect. The composition may therefore be administered as a single dose or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

### 5.6. Compositions and Kits

The invention provides compositions and kits for detecting and/or measuring types and levels of a particular analyte, e.g., a compound or biologic of interest, using the antibodies described herein. Kits for carrying out the diagnostic assays of the invention typically include, a suitable container means, (i) a probe that comprises an antibody or nucleic acid sequence that specifically binds to the marker polypeptides or polynucleotides of the invention; (ii) a label for detecting the presence of the probe; and (iii) instructions for how to measure the analyte. The kits may include several antibodies of the invention, e.g., a first antibody and/or second and/or third and/or additional antibodies that recognize the analyte. The container means of the kits will generally include at least one vial, test tube, flask, bottle, syringe and/or other container into which a first antibody specific for one of the polypeptides or a first nucleic acid specific for one of the polynucleotides of the present invention may be placed and/or suitably aliquoted. Where a second and/or third and/or additional component is provided, the kit will also generally contain a second, third and/or other additional container into which this component may be placed. Alternatively, a container may contain a mixture of more than one antibody, each reagent specifically binding a different marker in accordance with the present invention. The kits of the present invention will also typically include means for containing the antibody or nucleic acid probes in close confinement for commercial sale. Such containers may include injection and/or blow-molded plastic containers into which the desired vials are retained.

The kits may further comprise positive and negative controls, as well as instructions for the use of kit components contained therein, in accordance with the methods of the present invention.

### 5.7. In Vivo Imaging

The antibodies of the invention also provide reagents for in vivo imaging such as, for instance, the imaging of a particular analyte associated with a particular disease using labeled antibodies. In vivo imaging techniques may be used, for example, as guides for surgical resection or to detect the distant spread of a particular cancer. For in vivo imaging purposes, reagents that detect the presence of these proteins or genes, such as antibodies, may be labeled with a positron-emitting isotope (e.g., 18F) for positron emission tomography (PET), gamma-ray isotope (e.g., 99mTc) for single photon emission computed tomography (SPECT), a paramagnetic molecule or nanoparticle (e.g., Gd³⁺ chelate or coated magnetite nanoparticle) for magnetic resonance imaging (MRI), a near-infrared fluorophore for near-infra red (near-IR) imaging, a luciferase (firefly, bacterial, or coelenterate), green fluorescent protein, or other luminescent molecule for bioluminescence imaging, or a perfluorocarbon-filled vesicle for ultrasound.

Furthermore, such labeled antibodies may include a fluorescent moiety, such as a fluorescent protein, peptide, or fluorescent dye molecule. Common classes of fluorescent dyes include, but are not limited to, xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Fluorescent dyes are discussed, for example, in U.S. Pat. No. 4,452,720 (Harada et al.); U.S. Pat. No. 5,227,487 (Haugland and Whitaker); and U.S. Pat. No. 5,543,295 (Bronstein et al.). Other fluorescent labels suitable for use in the practice of this invention include a fluorescein dye. Typical fluorescein dyes include, but are not limited to, 5-carboxyfluorescein, flu orescein-5-isothiocyanate, and 6-carboxyfluorescein; examples of other fluorescein dyes can be found, for example, in U.S. Pat. No. 4,439,356 (Khanna and Colvin); U.S. Pat. No. 5,066,580 (Lee), U.S. Pat. No. 5,750,409 (Hermann et al.); and U.S. Pat. No. 6,008,379 (Benson et al.).

The kits may include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®, and other rhodamine dyes. Other rhodamine dyes can be found, for example, in U.S. Pat. No. 5,936,087 (Benson et al.), U.S. Pat. No. 6,025,505 (Lee et al.); U.S. Pat. No. 6,080,852 (Lee et al.). The kits may include a cyanine dye, such as, for example, Cy3, Cy3B, Cy3.5, Cy5, Cy5.5, Cy7. Phosphorescent compounds including porphyrins, phthalocyanines, polyaromatic compounds such as pyrenes, anthracenes and acenaphthenes, and so forth, may also be used.

Unless defined otherwise, all technical and scientific terms used herein have the same meaning as commonly understood by one of ordinary skill in the art to which this invention belongs. The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention. In particular, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### 6. EXAMPLES

### 6.1. B Cell Analysis

Antibodies are produced by recombined genomic immunoglobulin (Ig) sequences in B lineage cells. Immunoglobulin light chains are derived from either κ or λ genes. The λ genes are comprised of four constant (C) region genes and approximately thirty variable (V) region genes. In contrast, the κ genes are comprised of one C region gene and 250 V region genes. The heavy chain gene family is comprised of several hundred V gene segments, fifteen D gene segments, and four joining (J) gene segments. Somatic recombination during B cell differentiation randomly chooses one V-D-J combination in the heavy chain and one V-J combination in either κ or λ light chain. Because there are so many gene segments, millions of unique combinations are possible. The V regions also undergo somatic hypermutation after recombination, generating further diversity. Despite this underlying complexity, it is possible to use dozens of primers targeting conserved sequences to sequence the full heavy and light chain complement in several multiplexed reactions (van Dongen et al., 2003 Leukemia 17: 2257-2317).

The vast majority of diversity in the B cell repertoire is comprised of the V-D-J regions of IgII and V-J regions of Igκ (Sandberg et al., 2005 Journal of Molecular Diagnostics 7:495-503; Boyd et al., 2009 Science Translational Med 1:12ra23). Previously-reported primer pools (van Dongen et al., 2003 Leukemia 17: 2257-2317) are used to amplify these regions of IgH and Igκ. Five primer pools in separate reactions are used to amplify the IgH and Igκ complement of a healthy human. The amplified material sequenced with bulk sequencing. To analyze the bulk sequencing results, the IgBLAST algorithm and database is used to determine the V-D and D-J junctions of IgH and align the IgH and Iκ sequences to germ line gene segments. Overall, this method is more highly parallelized than previously-reported methods for single cell Ig analysis (U.S. Pat. No. 7,749,697).

### 6.2. B Cell Analysis and Drug Discovery

Antibody therapeutics are increasingly used by pharmaceutical companies to treat intractable diseases such as cancer (Carter 2006 Nature Reviews Immunology 6:343-357; Carter 2011 Experimental Cell Res 317 1261-1269; Reichart 2011 mAbs 3:1 76-99; Reichart 2010 mAbs 2:6 695-700). The current process of antibody drug discovery is expensive and tedious, requiring the identification of an antigen, and then the isolation and production of monoclonal antibodies with activity against the antigen. Individuals that have been exposed to disease produce antibodies against antigens associated with that disease, so it is possible mine patient immune repertoires for antibodies that could be used for pharmaceutical development. Patient immune repertoires offer vastly greater diversity than antibodies associated with a specific antigen over-expressed in a particular disease or disorder. However, a functional monoclonal antibody requires both heavy and light chain immunoglobulins.

Recently some researchers have reported methods to isolate antibody sequences directly from an organism. Specifically, Boyd et al. used multiplexed amplification of IgII coupled with bulk sequencing to survey the antibody complement of human subjects (Boyd et al., 2009, Sci Transl Med 1: 12ra23). Cheung et al. recently reported the proteomic identification of sequences associated antibodies having desired binding activity and use of those sequences to datamine B-cell immune repertoires (Cheung et al. 2012 Nat Biotech 30(5) 447-452). Reddy et al. disclosed a method of direct isolation of antibodies from immunized mice by pairing the most abundant variable heavy (V_{H}) and variable light (V_{L}) genes (Reddy et al. 2010 Nat Biotech 28(9) 965-969).

Exemplary PCR primers for multiplexed amplification of the full IgH repertoire are included in Table 1. Fusion primers and linker primers for human and mouse sequences are also shown in Sections 6.3.1 and 6.3.2 below. See also, Meijer et al. 2009 “Human Antibody Repertoires” in Therapeutic Antibodies: Methods and Protocols vol. 525, A. S. Dimitrov ed. Chap. 13, p. 261-277; Meijer et al. 2006 J Mol Biol 358 764-772; U.S. Pat. No. 7,749,697 (Olekiewicz et al.).

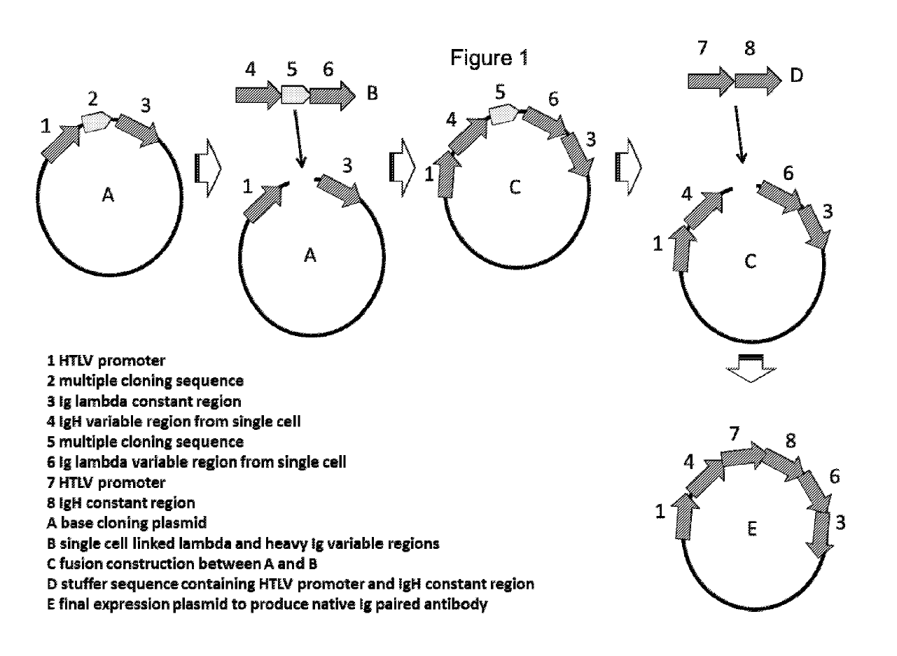

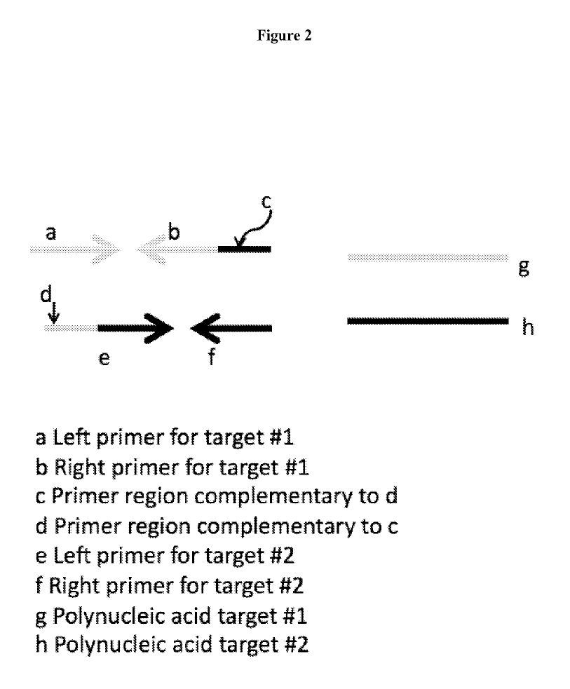

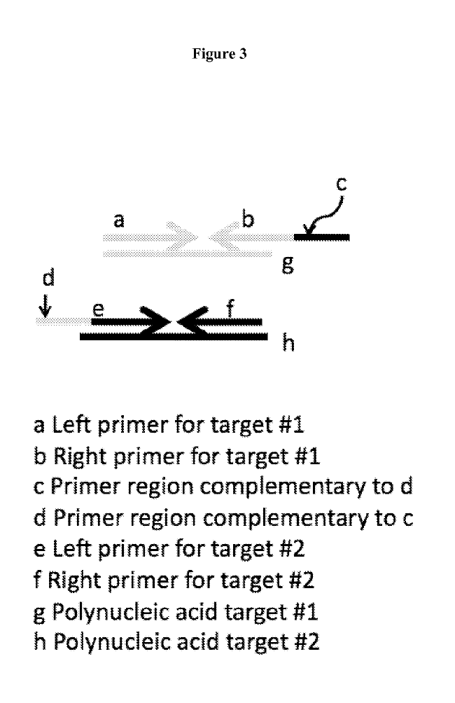

FIG. 1 shows a schematic of how to practice the invention. Specifically, it shows how the fusion primers are used to connect pieces 4 and 6, where 5 is the fusion or linker region (described in FIGS. 2 and 3 as sequences c and d). A set of nucleic acid probes for the invention may contain, for example, (i) an IgHV or IgHJ primer as a first probe, (ii) an IgHJ fusion or IgHV fusion primer as a second probe, (iii) an IgLJ fusion or IgLV fusion primer as a third probe, and (iv) an IgLV or IgLJ primer as the fourth probe. Amplifiable and fused product can be built in multiple combinations using the methods of the invention. The first and second probes amplify one target, e.g., IgH variable region, while probes three and four amplify a second target, e.g., IgL variable region.

In one embodiment of the invention, the linked variable regions are then subcloned into a protein expression plasmid using restriction enzymes (Sequence Λ in FIG. 1). The protein expression plasmid contains a promoter sequence and a light chain Ig constant sequence. In order to express the full light and heavy Ig protein sequences, a second subcloning step must be carried out. In this subcloning step, a second promoter and a heavy chain Ig constant region (Sequence D in FIG. 1) are subcloned into the ligation product of the original plasmid and the fused variable regions (Sequence C in FIG. 1). The final product (Sequence E) is an expression vector that contains DNA coding sequence for expression of both heavy and light chain Ig in cellular systems such as phage display, yeast display, or mammalian display. This step enables downstream functional analysis or use of the antibodies, i.e., screening the antibodies for antigen affinity, and/or production of paired-chain antibodies for experimental, diagnostic, or therapeutic use.

In an embodiment, one of either of the first or second probes is a fusion primer. Similarly, one of either the third or fourth probes is a fusion primer. The order of V primer+J fusion primer as probes one (three) and two (four) or J primer+V fusion-primer as probes one (three) and two (four) is arbitrary. Primers for all possible combinations and two species (homo sapiens and mus musculus) are listed below in Sections 6.3.1 and 6.3.2, respectively.

Primer design is largely based on the scope of the project, the laboratory techniques, the sequencing technology, and the data analysis method. Primers are selected to target the nucleic acid sequence of interest, minimize misidentification. Fusion sequences are designed with maximum possible Hamming distance to the best alignment with the target nucleic acid sequence of interest. Hamming, 1950, Bell System Tech. J. 29: 147-160). One of ordinary skill may readily obtain additional sequences for primer design from databases such as RefSeq (http://www.ncbi.nlm.nih.gov/gene/), the international ImMunoGeneTics Information System® (http://www.imgt.org/), EMBL Nucleotide Sequence Database VBASE2 (http://www.vbase2.org/), or MRC Centre for Protein Engineering V BASE (http://vbase.mrc-cpe.cam.ac.uk/).

In one embodiment of the invention, single heavy or light chain Ig is surveyed by multiplexed sequencing of samples from a clinically relevant site, e.g., in a tumor or in lymph tissue near the tumor. Such tissues are known to contain B cells and other types of antibody-producing cells (Hansen et al., 2001, PNAS 98:12659-12554).

In one embodiment of the invention, overlap extension PCR and/or overlap extension RT-PCR in single cell emulsion microdroplets is used to capture functional antibody sequences from subject B cell repertoires. Briefly, the method involves the following steps: (i) isolation of single B cells in aqueous-in-oil microreactors using a microfluidic device; (ii) molecular linkage between heavy and light chain immunoglobulin (e.g., IgH and Igκ) amplicons inside the single cell microreactors; and (iii) reversal of the emulsions followed by bulk sequencing of the linked polynucleic acid sequences. This produces heavy and light chain pairings from thousands to hundreds of thousands to millions of single B cells analyzed in parallel.

In this embodiment, the fusion primer sequences for overlap extension PCR and overlap extension RT-PCR are identical to the independent IgH and Igκ primers, except certain primers contain additional polynucleotide sequences for overlap extension: (i) the forward primer of the IgII locus has a random 10-20 nt sequence with no complementarity to either target; (ii) the reverse primer of the IgH loci has a 10-20 nt sequence with complementarity to the forward primer of Igκ; and (iii) the forward primer of Igκ has complementarity to the reverse primers for the IgH locus. In the final reaction mixtures, the outer primers are diluted to a final concentration of 0.1 μM, and the inner primers are diluted to 0.01 μM, such that the inner primers will be a limiting reagent. This drives formation of the major amplicon.

Finally, in this embodiment of the invention, we use a computer algorithm to integrate the blood and target tissue data sets. Briefly, we identify the most frequent heavy or light chain Ig clones in the target tissue. In one embodiment, the 10 most frequent heavy or light chain Ig clones are chosen. Next, we mine the overlap extension PCR data for said clones that are most frequent in the target tissue. Because the overlap extension PCR data retains native Ig pairings, we identify the fully functional target tissue-directed antibody and then physically clone the antibodies for functional assessment. Said functional assessment may include ELISA, antigen affinity assays, and immunogenicity studies.

In another embodiment, single heavy and/or light chain Ig is surveyed by multiplexed sequencing of samples from a clinically relevant site, e.g., in a tumor or in lymph tissue near the tumor. A list of candidate fully paired antibodies are then identified computationally by an all-by-all pairing of the oligoclonal heavy chain repertoire with the oligoclonal light chain repertoire. For example, in certain embodiments the 10 most common heavy chains and the ten most common light chains are selected from repertoire sequencing data. These heavy and light chains then produce a matrix of 100 heavy and light chain pairs. These 100 pairs can then be inserted into antibody expression vectors and screened for antigen affinity and/or immunogenicity. Whereas prior methods have computationally paired heavy and light chain Ig in immunized mice (Reddy et al. 2010 Nat Biotech 28(9) 965-969), our method uses only native heavy and light chain Ig. In particular, in the current invention, native heavy and light chain Ig from diseased individuals are used to generate a matrix of 100 heavy and light chain pairs.

In another embodiment of the invention, Ig repertoires are analyzed longitudinally in single individuals or populations of individuals that share traits, and oligoclonal B cell populations are correlated with an immune response to a disease or condition. In one embodiment, computer algorithms are used to select the 10 most common heavy chains and the 10 most common light chains present in repertoire sequencing data from the samples collected at the time of the disease or condition but not present in the repertoire sequencing data from the sample at the time without the disease or condition. These heavy and light chains then produce a matrix of 100 heavy and light chain pairs. These 100 pairs can then be inserted into antibody expression vectors and screened for antigen affinity and/or immunogenicity. In other embodiments, the top 1% of heavy chain Ig clones that are present in samples with said disease or condition but not in the sample without said disease or condition are paired with the top 1% of light chain Ig clones that are present in samples with said disease or condition but not in the sample without said disease or condition. Said analysis is possible for single individuals tested at two time points, in groups of individuals that share the same traits and tested at two time points, or in at least two groups of individuals that represent “healthy” and “disease” populations.

In other embodiments, T cells are used instead of B cells, with the goal of discovering functional TCR rather than antibodies. The functional TCR can then be used to engineer therapeutic T cells for diseases such as cancer.

### 6.3. Exemplary PCR and Fusion Primer Sequences

### 6.3.1. Human (homo sapiens) Ig

TABLE 1
Primers for multiplexed amplification of human IgH
(see Figure 2 and 3 primers a or f).

| | |
|---|---|
| 1209F_IgH_human | CAGTTCTCCCTGAAGCTGAGCT |
| 1210F_IgH_human | CAGCCTACATGGAGCTGAGCA |
| 1211F_IgH_human | ACACGCTGTATCTGCAAATGAACA |
| 1212F_IgH_human | GAACTCACTGTATCTGCAAATGAACA |
| 1213F_IgH_human | GAACACGCTGTATCTTCAAATGAACA |
| 1214F_IgH_human | CTCCCTGTATCTGCAAATGAACAGT |
| 1215F_IgH_human | CAGGTGGTCCTTACAATGACCAAC |
| 1216F_IgH_human | GCCTACCTGCAGTGGAGCA |
| 1217F_IgH_human | GCAGACACGGCCGTGTATTA |
| 1218F_IgH_human | GCACGGCATATCTGCAGATCTG |
| 1219F_IgH_human | GAGCTGAGGACACGGCTGTGTAT |
| 1220F_IgH_human | GACACAGCCTACATGGAGCTGAG |
| 1221F_IgH_human | GAACACGCTGCATCTTCAAATGAA |
| 1222F_IgH_human | CGGCGTATCTGCAAATGAACA |
| 1223F_IgH_human | CGCTGTATCTTCAAATGGGCA |
| 1224F_IgH_human | CATCGCCTATCTGCAAATGAACA |
| 1225F_IgH_human | CAGTTCTCCCTGCAGCTGAACT |
| 1226F_IgH_human | CACTGCCTACCTGCAGTGGAG |
| 1227F_IgH_human | CACGGCATATCTGCAGATCTG |
| 1228F_IgH_human | CACAGTCTACATGGAGCTGAGCA |
| 1229F_IgH_human | CAAGAACTCCTTGTATCTTCAAATGAACA |
| 1230F_IgH_human | AGGTGGTCCTTACCATGACCAAC |
| 1231F_IgH_human | AGAACACGCTGTATCTGCAAATGAA |
| 1232F_IgH_human | ACAGCCTACATGGAGCTGAGGA |
| 1233F_IgH_human | AAGAACTCACTGTATCTGCAAATGAACA |
| 1234F_IgH_human | AACTCCCTGTATCTGCAAATGAACA |
| 1235F_IgH_human | AACACGCTGTATCTGCAAATGAACA |
| 1236R_IgH_human | ACCTGAGGAGACGGTGACC |

HSIgLJRC.primers.65C
>HSIgLJ1 64.22
GACCTTGGTCCCAGTTCCGAA
>HSIgLJ2 64.54
CTTGGTCCCTCCGCCGAA
>HSIgLJ3 65.29
CTTGGTCCCTCCGCCGAA
>HSIgLJ6 66.31
CCTTGGTGCCACTGCCGAA
>HSIgLJ7 66.68
GCTGGGTGCCTCCTCCGAA
HSIgLV.primers.65C
>HSIGLV1-36 65.32
GGGCTCCAGTCTGAGGATGAGG
>HSIGLV1-40 66.36
GGGCTCCAGTCTGAGGATGAGG
>HSIGLV1-44 65.32
GGGCTCCAGTCTGAGGATGAGG
>HSIGLV1-47 63.82
CTCCGGTCCGAGGATGAGG
>HSIGLV1-51 64.15
GACTCCAGACTGGGGACGAGG
>HSIGLV2-8 65.71
GGGCTCCAGGCTGAGGATGA
>HSIGLV2-11 65.71
TGACCATCTCTGGGCTCCAGGCTGAGGATGA
>HSIGLV2-14 64.75
GGCTCCAGGCTGAGGACGA
>HSIGLV3-1 66.25
AGCGGGACCCAGGCTATGGA
>HSIGLV3-9 65.97
CAGAGCCCAAGCCGGGG
>HSIGLV3-10 64.83
GGCCCAGGTGGAGGATGAAG
>HSIGLV3-12 64.96
GATCGAGGCTGGGGATGAGG
>HSIGLV3-16 64.83
TGAGTCCAGGCAGAAGACGAGG
>HSIGLV3-19 65.32
GCTCAGGCGGAAGATGAGGC
>HSIGLV3-21 62.50
GAAGCCGGGGATGAGGC
>HSIGLV3-22 63.97
CAGCAGGGTCCTGACCGAAG
>HSIGLV3-25 62.91
CAGAAGATGAGGCTGACTATT
>HSIGLV3-27 65.72
CGGGGCCCAGGTTGAGG
>HSIGLV4-3 65.23
CCAGTCTGACGATGAGGCTGAGTATC
>HSIGLV4-60 65.82
CCATCTCCAACCTCCAGTTTGAGG
>HSIGLV4-69 65.40
CATCTCCAGCCTCCAGTCTGAGG
>HSIGLV5-37 64.60
CCGGGCTCCAGTCTGAGGA
>HSIGLV5-39 64.93
CATCTCTGGGCTCCAGTCTGAAGA
>HSIGLV5-45 64.51
ATCTCTGGGCTCCAGTCTGAGGA
>HSIGLV5-52 63.13
GCTCCAGCCTGAGGATGAGG
>HSIGLV6-57 63.94
CCATCTCTGGACTGAAGACTGAGGA
>HSIGLV7-43 64.65
GTCAGGTGTGCAGCCTGAGGA
>HSIGLV7-46 63.23
GGTGCGCAGCCTGAGGA
>HSIGLV8-61 63.40
GGGCCCAGGCAGATGATG
>HSIGLV9-49 61.57
CCAGGAAGAAGATGAGAGTGACTACC
>HSIGLV10-54 64.39
GACTCCAGCCTGAGGACGAGG
HSIgHV.fusion.primers
>IgHV1-2
CCCGAGCTCATCTGGCATAATTCTCCTAGTTTTCCAGCCTACATGGAGCTGAGCA
>IgHV1-3
CCCGAGCTCATCTGGCATAATTCTCCTAGTTTTCCAGCCTACATGGAGCTGAGCA
>IgHV1-8
CCCGAGCTCATCTGGCATAATTCTCCTAGTTTTCCAGCCTACATGGAGCTGAGCA
>IgHV1-f
CCCGAGCTCATCTGGCATAATTCTCCTAGTTTTCGACACAGCCTACATGGAGCTGAG
>IgHV1-18
CCCGAGCTCATCTGGCATAATTCTCCTAGTTTTCACAGCCTACATGGAGCTGAGGA
>IgHV1-24
CCCGAGCTCATCTGGCATAATTCTCCTACTTTTCCAGCCTACATGGAGCTGAGCA
>IgHV1-45
CCCGAGCTCATCTGGCATAATTCTCCTAGTTTTCCAGCCTACATGGAGCTGAGCA
>IgHV1-46
CCCGAGCTCATCTGGCATAATTCTCCTACTTTTCCACAGTCTACATGGAGCTGAGCA
>IgHV1-58
CCCGAGCTCATCTGGCATAATTCTCCTAGTTTTCCAGCCTACATGGAGCTGAGCA
>IgHV1-69
CCCGAGCTCATCTGGCATAATTCTCCTAGTTTTCCAGCCTACATGGAGCTGAGCA
>IgHV2-5
CCCGAGCTCATCTGGCATAATTCTCCTAGTTTTCCAGGTGGTCCTTACAATGACCAAC
>IgHV2-26
CCCGAGCTCATCTGGCATAATTCTCCTAGTTTTCAGGTGGTCCTTACCATGACCAAC
>IgHV2-70
CCCGAGCTCATCTGGCATAATTCTCCTAGTTTTCCAGGTGGTCCTTACAATGACCAAC
>IgHV3-d
CCCGAGCTCATCTGGCATAATTCTCCTAGTTTTCGAACACGCTGCATCTTCAAATGAA
>IgHV3-NL1
CCCGAGCTCATCTGGCATAATTCTCCTAGTTTTCGAGCTGAGGACACGGCTGTGTAT
>IgHV3-9
CCCGAGCTCATCTGGCATAATTCTCCTAGTTTTCCTCCCTGTATCTGCAAATGAACAGT
>IgHV3-11
CCCGAGCTCATCTGGCATAATTCTCCTAGTTTTCAAGAACTCACTGTATCTGCAAATGAACA
>IgHV3-13
CCCGAGCTCATCTGGCATAATTCTCCTAGTTTTCCAAGAACTCCTTGTATCTTCAAATGAACA
>IgHV3-15
CCCGAGCTCATCTGGCATAATTCTCCTAGTTTTCAACACGCTGTATCTGCAAATGAACA
>IgHV3-20
CCCGAGCTCATCTGGCATAATTCTCCTAGTTTTCAACTCCCTGTATCTGCAAATGAACA
>IgHV3-21
CCCGAGCTCATCTGGCATAATTCTCCTAGTTTTCGAACTCACTGTATCTGCAAATGAACA
>IgHV3-23
CCCGAGCTCATCTGGCATAATTCTCCTAGTTTTCACACGCTGTATCTGCAAATGAACA
>IgHV3-30
CCCGAGCTCATCTGGCATAATTCTCCTAGTTTTCACACGCTGTATCTGCAAATGAACA
>IgHV3-30-3
CCCGAGCTCATCTGGCATAATTCTCCTAGTTTTCAGAACACGCTGTATCTGCAAATGAA
>IgHV3-33
ACCCGAGCTCATCTGGCATAATTCTCCTAGTTTTCCACGCTGTATCTGCAAATGAACA
>IgHV3-43
CCCGAGCTCATCTGGCATAATTCTCCTAGTTTTCCTCCCTGTATCTGCAAATGAACAGT
>IgHV3-48
GCCCGAGCTCATCTGGCATAATTCTCCTAGTTTTCAACTCACTGTATCTGCAAATGAACA
>IgHV3-49
CCCGAGCTCATCTGGCATAATTCTCCTAGTTTTCCATCGCCTATCTGCAAATGAACA
>IgHV3-53
CCCGAGCTCATCTGGCATAATTCTCCTAGTTTTCGAACACGCTGTATCTTCAAATGAACA
>IgHV3-64
CCCGAGCTCATCTGGCATAATTCTCCTAGTTTTCCGCTGTATCTTCAAATGGGCA
>IgHV3-66
CCCGAGCTCATCTGGCATAATTCTCCTACTTTTCGAACACGCTGTATCTTCAAATGAACA
>IgHV3-72
CCCGAGCTCATCTGGCATAATTCTCCTAGTTTTCGAACTCACTGTATCTGCAAATGAACA
>IgHV3-73
CCCGAGCTCATCTGGCATAATTCTCCTAGTTTTCCGGCGTATCTGCAAATGAACA
>IgHV3-74
CCCGAGCTCATCTGGCATAATTCTCCTAGTTTTCACACGCTGTATCTGCAAATGAACA
>IgHV4-b
CCCGAGCTCATCTGGCATAATTCTCCTAGTTTTCCACGGCATATCTGCAGATCTG
>IgHV4-4
CCCGAGCTCATCTGGCATAATTCTCCTAGTTTTCCAGTTCTCCCTGAAGCTGAGCT
>IgHV4-28
CCCGAGCTCATCTGGCATAATTCTCCTAGTTTTCCAGTTCTCCCTGAAGCTGAGCT
>IgHV4-30-2
CCCGAGCTCATCTGGCATAATTCTCCTAGTTTTCCAGTTCTCCCTGAAGCTGAGCT
>IgHV4-30-4
CCCGAGCTCATCTGGCATAATTCTCCTAGTTTTCGCAGACACGGCCGTGTATTA
>IgHV4-31
CCCGAGCTCATCTGGCATAATTCTCCTAGTTTTCCAGTTCTCCCTGAAGCTGAGCT
>IgHV4-34
CCCGAGCTCATCTGGCATAATTCTCCTAGTTTTCCAGTTCTCCCTGAAGCTGAGCT
>IgHV4-39
CCCGAGCTCATCTGGCATAATTCTCCTAGTTTTCCAGTTCTCCCTGAAGCTGAGCT
>IgHV4-59
CCCGAGCTCATCTGGCATAATTCTCCTAGTTTTCCAGTTCTCCCTGAAGCTGAGCT
>IgHV4-61
CCCGAGCTCATCTGGCATAATTCTCCTAGTTTTCCAGTTCTCCCTGAAGCTGAGCT
>IgHV5-a
CCCGAGCTCATCTGGCATAATTCTCCTAGTTTTCCACTGCCTACCTGCAGTGGAG
>IgHV5-51
CCCGAGCTCATCTGGCATAATTCTCCTAGTTTTCGCCTACCTGCAGTGGAGCA
>IgHV6-1
CCCGAGCTCATCTGGCATAATTCTCCTAGTTTTCCAGTTCTCCCTGCAGCTGAACT
>IgHV7-4-1
CCCGAGCTCATCTGGCATAATTCTCCTAGTTTTCGCACGGCATATCTGCAGATCTG
HSIgHJ.fusionprimers
>HSIgHJAll, universal
CCCGAGCTCATCTGGCATAATTCTCCTAGTTTTCACCTGAGGAGACGGTGACC
HSIgLJRC.fusion.primers
>HSIgLJ1
GAAAACTAGGAGAATTATGCCAGATGAGCTCGGGGGACCTTGGTCCCAGTTCCGAA
>HSIgLJ2
GAAAACTAGGAGAATTATGCCAGATGAGCTCGGGGCTTGGTCCCTCCGCCGAA
>HSIgLJ3
GAAAACTAGGAGAATTATGCCAGATGAGCTCGGGGCTTGGTCCCTCCGCCGAA
>HSIgLJ6
GAAAACTAGGAGAATTATGCCAGATGAGCTCGGGGCCTTGGTGCCACTGCCGAA
>HSIgLJ7
GAAAACTAGGAGAATTATGCCAGATGAGCTCGGGGGCTGGGTGCCTCCTCCGAA
HSIgLV.fusion.primers
>HSIGLV1-36
GAAAACTAGGAGAATTATGCCAGATGAGCTCGGGGGGCTCCAGTCTGAGGATGAGG
>HSIGLV1-40
GAAAACTAGGAGAATTATGCCAGATGAGCTCGGGGGGCTCCAGGCTGAGGATGACG
>HSIGLV1-44
GAAAACTAGGAGAATTATGCCAGATGAGCTCGGGGGGCTCCAGTCTGAGGATGAGG
>HSIGLV1-47
GAAAACTAGGAGAATTATGCCAGATGAGCTCGGGCTCCGCTCCGAGGATGAGG
>HSIGLV1-51
GAAAACTAGGAGAATTATGCCAGATGAGCTCGGGGACTCCAGACTGGGGACGAGG
>HSIGLV2-8
GAAAACTAGGAGAATTATGCCAGATGAGCTCGGGGGGCTCCAGGCTGAGGATGA
>HSIGLV2-11
GAAAACTAGGAGAATTATGCCAGATGAGCTCGGGTGACCATCTCTGGGCTCCAGGCTGAGGATGA
>HSIGLV2-14
GAAAACTAGGAGAATTATGCCAGATGAGCTCGGGGGCTCCAGGCTGAGGACGA
>HSIGLV3-1
GAAAACTAGGAGAATTATGCCAGATGAGCTCGGGAGCGGGACCCAGGCTATGGA
>HSIGLV3-9
GAAAACTAGGAGAATTATGCCAGATGAGCTCGGGCAGAGCCCAAGCCGGGG
>HSIGLV3-10
GAAAACTAGGAGAATTATGCCAGATGAGCTCGGGGGCCCAGGTGGAGGATGAAG
>HSIGLV3-12
GAAAACTAGGAGAATTATGCCAGATGAGCTCGGGGATCGAGGCTGGGGATGAGG
>HSIGLV3-16
GAAAACTAGGAGAATTATGCCAGATGAGCTCGGGTGAGTCCAGGCAGAAGACGAGG
>HSIGLV3-19
GAAAACTAGGAGAATTATGCCAGATGAGCTCGGGGCTCAGGCGGAAGATGAGGC
>HSIGLV3-21
GAAAACTAGGAGAATTATGCCAGATGAGCTCGGGGAAGCCGGGGATGAGGC
>HSIGLV3-22
GAAAACTAGGAGAATTATGCCAGATGAGCTCGGGCAGCAGGGTCCTGACCGAAG
>HSIGLV3-25
GAAAACTAGGAGAATTATGCCAGATGAGCTCGGGCAGAAGATGAGGCTGACTATT
>HSIGLV3-27
GAAAACTAGGAGAATTATGCCAGATGAGCTCGGGCGGGGCCCAGGTTGAGG
>HSIGLV4-3
GAAAACTAGGAGAATTATGCCAGATGAGCTCGGGCCAGTCTGACGATGAGGCTGAGTATC
>HSIGLV4-60
GAAAACTAGGAGAATTATGCCAGATGAGCTCGGGCCATCTCCAACCTCCAGTTTGAGG
>HSIGLV4-69
GAAAACTAGGAGAATTATGCCAGATGAGCTCGGGCATCTCCAGCCTCCAGTCTGAGG
>HSIGLV5-37
GAAAACTAGGAGAATTATGCCAGATGAGCTCGGGCCGGGCTCCAGTCTGAGGA
>HSIGLV5-39
GAAAACTAGGAGAATTATGCCAGATGAGCTCGGGCATCTCTGGGCTCCAGTCTGAAGA
>HSIGLV5-45
GAAAACTAGGAGAATTATGCCAGATGAGCTCGGGATCTCTGGGCTCCAGTCTGAGGA
>HSIGLV5-52
GAAAACTAGGAGAATTATGCCAGATGAGCTCGGGGCTCCAGCCTGAGGATGAGG
>HSIGLV6-57
GAAAACTAGGAGAATTATGCCAGATGAGCTCGGGCCATCTCTGGACTGAAGACTGAGGA
>HSIGLV7-43
GAAAACTAGGAGAATTATGCCAGATGAGCTCGGGGTCAGGTGTGCAGCCTGAGGA
>HSIGLV7-46
GAAAACTAGGAGAATTATGCCAGATGAGCTCGGGGGTGCGCAGCCTGAGGA
>HSIGLV8-61
GAAAACTAGGAGAATTATGCCAGATGAGCTCGGGGGGCCCAGGCAGATGATG
>HSIGLV9-49
GAAAACTAGGAGAATTATGCCAGATGAGCTCGGGCCAGGAAGAAGATGAGAGTGACTACC
>HSIGLV10-54
GAAAACTAGGAGAATTATGCCAGATGAGCTCGGGGACTCCAGCCTGAGGACGAGG
6.3.2. Mouse (mus musculus) Ig
MMIgLV.primers.65C
>MMIGLV1 64.50
CACAGGGGCACAGACTGAGGA
>MMIGLV2 63.88
CATCACAGGGGCACAGACTGAG
>MMIGLV3 62.12
ATCCAGCCTGAAGATGAAGCAATAT
MMIgLJRC.primers.65C
MMIgLJRC.primers.65C
>MMIgLJ1 63.00
CAGTTTGGTTCCTCCACCGAA
>MMIgLJ2 63.89
CTTGGTTCCACCGCCGAA
>MMIgLJ3 63.60
ACCTTGGTTCCACTGCCGAA
MMIgHJRC.fusion.primers
>MMIgHJ1
CCCGAGCTCATCTGGCATAATTCTCCTAGTTTTCGTGACCGTGGTCCCTGTGCCCCA
>MMIgHJ2
CCCGAGCTCATCTGGCATAATTCTCCTAGTTTTCGTGAGAGTGGTGCCTTGGCCCCA
>MMIgHJ3
CCCGAGCTCATCTGGCATAATTCTCCTAGTTTTCCAGAGTCCCTTGGCCCCA
>MMIgHJ4
CCCGAGCTCATCTGGCATAATTCTCCTAGTTTTCTGACTGAGGTTCCTTGACCCCA
MMIgHV.fusion.primers
>MMIgHV1-4
CCCGAGCTCATCTGGCATAATTCTCCTAGTTTTCAGCACAGCCTACATGCAACTGAG
>MMIgHV1-5
CCCGAGCTCATCTGGCATAATTCTCCTAGTTTTCAGCACTGCCTACATGGAGCTCAG
>MMIgHV1-7
CCCGAGCTCATCTGGCATAATTCTCCTAGTTTTCAGCACAGCCTACATGCAGCTGAG
>MMIgHV1-9
CCCGAGCTCATCTGGCATAATTCTCCTAGTTTTCCAACACAGCCTACATGCAACTCAG
>MMIgHV1-11
CCCGAGCTCATCTGGCATAATTCTCCTAGTTTTCCAGCACAGTGTACATGGTGTTGA
>MMIgHV1-12
CCCGAGCTCATCTGGCATAATTCTCCTAGTTTTCAGCACAGCCTACATGCAGCTCAG
>MMIgHV1-14
CCCGAGCTCATCTGGCATAATTCTCCTAGTTTTCAGCACAGCCTACATGGAGCTCAG
>MMIgHV1-15
CCCGAGCTCATCTGGCATAATTCTCCTAGTTTTCAGCACAGCCTACATGGAGCTCCG
>MMIgHV1-17-1
CCCGAGCTCATCTGGCATAATTCTCCTAGTTTTCACAGCCTATATGCAATTCAG
>MMIgHV1-18
CCCGAGCTCATCTGGCATAATTCTCCTAGTTTTCACAGCCTACATGGAGCTGAGGA
>MMIgHV1-19
CCCGAGCTCATCTGGCATAATTCTCCTAGTTTTCAGCACAGCCTACATGGAGCTCAA
>MMIgHV1-20
CCCGAGCTCATCTGGCATAATTCTCCTAGTTTTCAGCCCACATGGAGCTCCG
>MMIgHV1-22
CCCGAGCTCATCTGGCATAATTCTCCTAGTTTTCAGCACAGCCTACATGGAGCTCCG
>MMIgHV1-26
CCCGAGCTCATCTGGCATAATTCTCCTAGTTTTCAGCACAGCCTACATGGAGCTCCG
>MMIgHV1-31
CCCGAGCTCATCTGGCATAATTCTCCTAGTTTTCAGCACAGCCTACATGGAGCTCCG
>MMIgHV1-34
CCCGAGCTCATCTGGCATAATTCTCCTAGTTTTCAGCACAGCCTACATGGAGCTCCG
>MMIgHV1-36
CCCGAGCTCATCTGGCATAATTCTCCTAGTTTTCAGCACAGCCTACATGGAGCTATA
>MMIgHV1-37
CCCGAGCTCATCTGGCATAATTCTCCTAGTTTTCACAGCCCACATGGAGCTCCTAT
>MMIgHV1-39
CCCGAGCTCATCTGGCATAATTCTCCTACTTTTCAGCACAGCCTACATGCAGCTCAA
>MMIgHV1-42
CCCGAGCTCATCTGGCATAATTCTCCTAGTTTTCAGCACAGCCTACATGCAGCTCAA
>MMIgHV1-43
CCCGAGCTCATCTGGCATAATTCTCCTACTTTTCCTCTAGCACAGTCTACTTGGAGCTCAG
>MMIgHV1-47
CCCGAGCTCATCTGGCATAATTCTCCTAGTTTTCCTCTAGCACAGTCTACTTGGAGCTCAG
>MMIgHV1-49
CCCGAGCTCATCTGGCATAATTCTCCTAGTTTTCAGCACAGCCTACATGGAACTCAG
>MMIgHV1-50
CCCGAGCTCATCTGGCATAATTCTCCTAGTTTTCAGCACAGCCTACATGCAGCTCAG
>MMIgHV1-52
CCCGAGCTCATCTGGCATAATTCTCCTAGTTTTCAGCACAGCCTACATGCAGCTCAG
>MMIgHV1-53
CCCGAGCTCATCTGGCATAATTCTCCTAGTTTTCAAGTTCAAGAGCAAGGCCACACT
>MMIgHV1-54
CCCGAGCTCATCTGGCATAATTCTCCTAGTTTTCAGCACTGCCTACATGCAGCTCAG
>MMIgHV1-55
CCCGAGCTCATCTGGCATAATTCTCCTAGTTTTCAGAAGTTCAAGAGCAAGGCCACACT
>MMIgHV1-56
CCCGAGCTCATCTGGCATAATTCTCCTAGTTTTCAGCACAGCCTACATGCAGCTCAG
>MMIgHV1-58
CCCGAGCTCATCTGGCATAATTCTCCTAGTTTTCAGCACAGCCTACATGCAGCTCAG
>MMIgHV1-59
CCCGAGCTCATCTGGCATAATTCTCCTAGTTTTCAGCCTGACATCTGAGGACTCTGCG
>MMIgHV1-61
CCCGAGCTCATCTGGCATAATTCTCCTAGTTTTCAGCACAGCCTACATGCAGCTCAG
>MMIgHV1-62-1
CCCGAGCTCATCTGGCATAATTCTCCTAGTTTTCGACACATCCTCCAGCACAGCCTA
>MMIgHV1-62-2
CCCGAGCTCATCTGGCATAATTCTCCTAGTTTTCCCTCCAGCACAGTCTATATGGAGCTTAG
>MMIgHV1-62-3
CCCGAGCTCATCTGGCATAATTCTCCTAGTTTTCAGCACAGCCTACATGCAGCTCAG
>MMIgHV1-63
CCCGAGCTCATCTGGCATAATTCTCCTAGTTTTCAGCACAGCCTACATGCAGTTCAG
>MMIgHV1-64
CCCGAGCTCATCTGGCATAATTCTCCTAGTTTTCAGCACAGCCTACATGCAACTCAG
>MMIgHV1-66
CCCGAGCTCATCTGGCATAATTCTCCTAGTTTTCAGCACTGCCTACATGCAGCTCAG
>MMIgHV1-67
CCCGAGCTCATCTGGCATAATTCTCCTAGTTTTCAGCACAGCCTATATGGAACTTGC
>MMIgHV1-69
CCCGAGCTCATCTGGCATAATTCTCCTAGTTTTCAAAGTTCAAGGGCAAGTCCACATT
>MMIgHV1-71
CCCGAGCTCATCTGGCATAATTCTCCTAGTTTTCGCACAGTCTATATGGAGCTTAGTAGATTGA
>MMIgHV1-72
CCCGAGCTCATCTGGCATAATTCTCCTAGTTTTCAAGTTCAAGAGCAAGGCCACACT
>MMIgHV1-74
CCCGAGCTCATCTGGCATAATTCTCCTAGTTTTCGTTCAAGGGCAAGGCCACAT
>MMIgHV1-75
CCCGAGCTCATCTGGCATAATTCTCCTACTTTTCAGCACAGCCTACATGTTGCTCAG
>MMIgHV1-76
CCCGAGCTCATCTGGCATAATTCTCCTAGTTTTCAGCACAGCCTACATGCAGCTCAG
>MMIgHV1-77
CCCGAGCTCATCTGGCATAATTCTCCTAGTTTTCAGCACAGCCTACATGCAGCTCAG
>MMIgHV1-78
CCCGAGCTCATCTGGCATAATTCTCCTAGTTTTCAGCACAGCCTACATGCAGCTCAA
>MMIgHV1-80
CCCGAGCTCATCTGGCATAATTCTCCTACTTTTCAGCACAGCCTACATGCAGCTCAG
>MMIgHV1-81
CCCGAGCTCATCTGGCATAATTCTCCTAGTTTTCACAGCGTACATGGAGCTCCG
>MMIgHV1-82
CCCGAGCTCATCTGGCATAATTCTCCTACTTTTCAGCACAGCCTACATGCAACTCAG
>MMIgHV1-84
CCCGAGCTCATCTGGCATAATTCTCCTAGTTTTCAGCACAGCCTACATGCAGCTCAG
>MMIgHV1-85
CCCGAGCTCATCTGGCATAATTCTCCTAGTTTTCACAGCGTACATGGAGCTCCA
>MMIgHV2-2
CCCGAGCTCATCTGGCATAATTCTCCTAGTTTTCAAGAGCCAAGTTTTCTTTAAAATGAACA
>MMIgHV2-3
CCCGAGCTCATCTGGCATAATTCTCCTAGTTTTCAAGAGCCAAGTTTTCTTAAAACTGAACA
>MMIgHV2-4
CCCGAGCTCATCTGGCATAATTCTCCTAGTTTTCAAGAGCCAAGTTTTCTTTAAAATGAACA
>MMIgHV2-5
CCCGAGCTCATCTGGCATAATTCTCCTAGTTTTCAAGAGCCAAGTTTTCTTTAAAATGAACA
>MMIgHV2-6
CCCGAGCTCATCTGGCATAATTCTCCTAGTTTTCCAAGAGCCAAGTTTTCTTAAAAATGAA
>MMIgHV2-6-8
CCCGAGCTCATCTGGCATAATTCTCCTAGTTTTCAAGAGCCAAGTTTTCTTAAAAATGAACA
>MMIgHV2-7
CCCGAGCTCATCTGGCATAATTCTCCTAGTTTTCAAGAGCCAAGTTTTCTTTAAAATGAGCA
>MMIgHV2-9
CCCGAGCTCATCTGGCATAATTCTCCTAGTTTTCCAAGAGCCAAGTTTTCTTAAAAATGAAC
>MMIgHV2-9-1
CCCGAGCTCATCTGGCATAATTCTCCTAGTTTTCCAAGAGTCAAGTTTTCTTAAAAATGAACA
>MMIgHV3-1
CCCGAGCTCATCTGGCATAATTCTCCTAGTTTTCAAGAACCATTTCTTCCTGAAGTTGAA
>MMIgHV3-2
CCCGAGCTCATCTGGCATAATTCTCCTAGTTTTCGAACCAGTTCTCCCTGGAATTGAATT
>MMIgHV3-3
CCCGAGCTCATCTGGCATAATTCTCCTAGTTTTCGAACCAGTTCTCACTGAAGTTGAGTT
>MMIgHV3-4
CCCGAGCTCATCTGGCATAATTCTCCTAGTTTTCAGAACCAGTTATTCCTGCAGTTGAACT
>MMIgHV3-5
CCCGAGCTCATCTGGCATAATTCTCCTAGTTTTCAGAACCAGTTCTTCCTGGAAATGAACT
>MMIgHV3-6
CCCGAGCTCATCTGGCATAATTCTCCTAGTTTTCAAAACTCACTGTATCTGCAAATGAACA
>MMIgHV3-8
CCCGAGCTCATCTGGCATAATTCTCCTAGTTTTCAAGAACCAGTATTACCTGCAGTTGAA
>MMIgHV4-1
CCCGAGCTCATCTGGCATAATTCTCCTAGTTTTCAAAAATACGCTGTACCTGCAAATGAG
>MMIgHV4-2
CCCGAGCTCATCTGGCATAATTCTCCTAGTTTTCTACGCTGTACCTGCAAATGAGCA
>MMIgHV5-2
CCCGAGCTCATCTGGCATAATTCTCCTAGTTTTCACCCTGTACCTGCAAATGAGCA
>MMIgHV5-4
CCCGAGCTCATCTGGCATAATTCTCCTAGTTTTCAAGAACAACCTGTACCTGCAAATGAG
>MMIgHV5-6
CCCGAGCTCATCTGGCATAATTCTCCTACTTTTCGAACACCCTGTACCTGCAAATGAG
>MMIgHV5-9
CCCGAGCTCATCTGGCATAATTCTCCTAGTTTTCGAACACCCTGTACCTGCAAATGAG
>MMIgHV5-9-1
CCCGAGCTCATCTGGCATAATTCTCCTAGTTTTCGAACACCCTGTACCTGCAAATGAG
>MMIgHV5-12
CCCGAGCTCATCTGGCATAATTCTCCTAGTTTTCGAACACCCTGTACCTGCAAATGAG
>MMIgHV5-12-4
CCCGAGCTCATCTGGCATAATTCTCCTACTTTTCAGAACACCCTTTACCTGCAAATGAC
>MMIgHV5-15
CCCGAGCTCATCTGGCATAATTCTCCTAGTTTTCAGAACACCCTGTACCTGGAAATGAG
>MMIgHV5-16
CCCGAGCTCATCTGGCATAATTCTCCTACTTTTCCAAAGAACATTCTATACCTGCAAATGAG
>MMIgHV5-17
CCCGAGCTCATCTGGCATAATTCTCCTAGTTTTCAACACCCTGTTCCTGCAAATGAC
>MMIgHV6-3
CCCGAGCTCATCTGGCATAATTCTCCTAGTTTTCCCAAAAGTAGTGTCTACCTGCAAATGA
>MMIgHV6-4
CCCGAGCTCATCTGGCATAATTCTCCTAGTTTTCTTTCAAAAGTAGTGTCTACCTGCATATGAA
>MMIgHV6-5
CCCGAGCTCATCTGGCATAATTCTCCTAGTTTTCCCAAAAGTAGTGTCTACCTGCAAATGA
>MMIgHV6-6
CCCGAGCTCATCTGGCATAATTCTCCTAGTTTTCCCAAAAGTAGTGTCTACCTGCAAATGAA
>MMIgHV6-7
CCCGAGCTCATCTGGCATAATTCTCCTAGTTTTCGCATCCTCTATCTTCAAATGAACACACT
>MMIgHV7-1
CCCGAGCTCATCTGGCATAATTCTCCTAGTTTTCGCATCCTCTACCTTCAGATGAATGCCCT
>MMIgHV7-2
CCCGAGCTCATCTGGCATAATTCTCCTAGTTTTCGCATCCTCTATCTTCAAATGAACACACT
>MMIgHV7-3
CCCGAGCTCATCTGGCATAATTCTCCTAGTTTTCCATCCTCTATCTTCAAATGAATGCC
>MMIgHV7-4
CCCGAGCTCATCTGGCATAATTCTCCTAGTTTTCATCCTCTATCTTCAAATGAACACCCT
>MMIgHV8-2
CCCGAGCTCATCTGGCATAATTCTCCTAGTTTTCACCAAGTATTCCTTAAACTCACCAGTGT
>MMIgHV8-4
CCCGAGCTCATCTGGCATAATTCTCCTAGTTTTCCAACAACCAAGCATTCCTGAATAT
>MMIgHV8-5
CCCGAGCTCATCTGGCATAATTCTCCTAGTTTTCAACCAGGTATTCCTCAAGATCACCAGT
>MMIgHV8-6
CCCGAGCTCATCTGGCATAATTCTCCTAGTTTTCAACCAGGTATTCCTCAAGATCACCACT
>MMIgHV8-8
CCCGAGCTCATCTGGCATAATTCTCCTAGTTTTCAACCAGGTATTCCTCAAGATCGC
>MMIgHV8-11
CCCGAGCTCATCTGGCATAATTCTCCTAGTTTTCAGGTATTCCTCAAGATCGCCAGT
>MMIgHV8-12
CCCGAGCTCATCTGGCATAATTCTCCTAGTTTTCCCAGGTATTCCTCAAGATCACCAGT
>MMIgHV9-1
CCCGAGCTCATCTGGCATAATTCTCCTAGTTTTCAGCACTGCCTATTTGCAGATCAA
>MMIgHV9-2
CCCGAGCTCATCTGGCATAATTCTCCTAGTTTTCAGCACTGCCTATTTGCAGATCAA
>MMIgHV9-3
CCCGAGCTCATCTGGCATAATTCTCCTAGTTTTCAGCACTGCCTATTTGCAGATCAA
>MMIgHV9-4
CCCGAGCTCATCTGGCATAATTCTCCTAGTTTTCGCCAGCACTGCCTATTTACAGATAAG
>MMIgHV10-1
CCCGAGCTCATCTGGCATAATTCTCCTAGTTTTCGAAAGCATGCTCTATCTGCAAATGAA
>MMIgHV10-3
CCCGAGCTCATCTGGCATAATTCTCCTACTTTTCGCTCTATCTGCAAATGAACA
>MMIgHV11-1
CCCGAGCTCATCTGGCATAATTCTCCTAGTTTTCACCCTGTACCTGCAGATGAGCA
>MMIgHV11-2
CCCGAGCTCATCTGGCATAATTCTCCTAGTTTTCACCCTGTACCTGCAGATGAGCA
>MMIgHV12-3
CCCGAGCTCATCTGGCATAATTCTCCTAGTTTTCAGAACCAGTTCTTCCTCCAATTGAA
>MMIgHV13-1
CCCGAGCTCATCTGGCATAATTCTCCTAGTTTTCAGCAGTGCATACATGCAGATGAA
>MMIgHV13-2
CCCGAGCTCATCTGGCATAATTCTCCTAGTTTTCTCAAAAAGCAGTGTCTACCTAGAGATGAA
>MMIgHV14-1
CCCGAGCTCATCTGGCATAATTCTCCTAGTTTTCCACAGCCTACCTGCAGCTCAG
>MMIgHV14-2
CCCGAGCTCATCTGGCATAATTCTCCTAGTTTTCCACAGCCTACCTGCAGCTCAG
>MMIgHV14-3
CCCGAGCTCATCTGGCATAATTCTCCTAGTTTTCCACAGCCTACCTGCAGCTCAG
>MMIgHV14-4
CCCGAGCTCATCTGGCATAATTCTCCTAGTTTTCCACAGCCTACCTGCAGCTCAG
>MMIgHV15-2
CCCGAGCTCATCTGGCATAATTCTCCTAGTTTTCCAACACAGCCTACTTGGAGCTCAA
>MMIgHV16-1
CCCGAGCTCATCTGGCATAATTCTCCTAGTTTTCTTCTGAGAACTTATTGTATCTACAAATGAACA
MMIgLJRC.fusion.primers
>MMIgLJ1
GAAAACTAGGAGAATTATGCCAGATGAGCTCGGGCAGTTTGGTTCCTCCACCGAA
>MMIgLJ2
GAAAACTAGGAGAATTATGCCAGATGAGCTCGGGCTTGGTTCCACCGCCGAA
>MMIgLJ3
GAAAACTAGGAGAATTATGCCAGATGAGCTCGGGACCTTGGTTCCACTGCCGAA
MMIgLV.fusion.primers
>MMIGLV1
GAAAACTAGGAGAATTATGCCAGATGAGCTCGGGCACAGGGGCACAGACTGAGGA
>MMIGLV2
GAAAACTAGGAGAATTATGCCAGATGAGCTCGGGCATCACAGGGGCACAGACTGAG
>MMIGLV3
GAAAACTAGGAGAATTATGCCAGATGAGCTCGGGATCCAGCCTGAAGATGAAGCAATAT

While in the foregoing detailed description this invention has been described in relation to certain preferred embodiments thereof, and many details have been set forth for purposes of illustration, it will be apparent to those skilled in the art that the invention is susceptible to additional embodiments and that certain of the details described herein can be varied considerably without departing from the basic principles of the invention.

It also is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A method for discovering antibodies, comprising:
identifying single chain immunoglobulin sequences in a target tissue from an animal;
providing a first set of nucleic acid probes, the first set comprising a first probe comprising a sequence that is complementary to an immunoglobulin heavy chain sequence, a second probe comprising a sequence that is complementary to the immunoglobulin heavy chain sequence and a second sequence that is complementary to an exogenous sequence, a third probe comprising a sequence that is complementary to the portion of the second probe that is complementary to the exogenous sequence and a sequence that is complementary to an immunoglobulin light chain sequence, and a fourth probe comprising a sequence that is complementary to an immunoglobulin light chain sequence;
isolating single antibody-producing cells from said animal;
amplifying immunoglobulin heavy and light chain targets independently, wherein the heavy chain sequence is amplified using the first probe and the second probe, and wherein the light chain sequence is amplified using the third probe and the fourth probe;
generating a fused complex by hybridizing the complementary sequence regions of the amplified said heavy and light chain sequences and amplifying the hybridized sequences using the first and fourth probes;
performing a bulk sequencing reaction to generate sequence information for at least 100,000 fused complexes from at least 10,000 cells within the population of cells, wherein the sequence information is sufficient to co-localize the first target nucleic acid sequence and the second target nucleic acid sequence to a single cell from the population of at least 10,000 cells; and
computationally matching said oligoclonal single chain immunoglobulin sequences with single chains from said fused complexes to identify a candidate list of paired immunoglobulin sequences to which the target tissue may be antigenic.

2. The method of claim 1, wherein said cells are B cells, bone marrow plasma cells, or plasma cells.

3. The method of claim 1, wherein said animal is a mammal.

4. The method of claim 3, wherein said mammal is a human.

5. The method of claim 1, wherein said tissue is a tissue sampled from an animal with a disease selected from the group consisting of: multiple sclerosis, rheumatoid arthritis, ulcerative colitis, Crohn's disease, systemic lupus erythematosus, graft-versus-host-disease, and host-versus-graft disease.

6. The method of claim 1, wherein said tissue is a tumor selected from the group consisting of: lung carcinoma, non-small cell lung cancer, small cell lung cancer, uterine cancer, thyroid cancer, breast carcinoma, prostate carcinoma, pancreas carcinoma, colon carcinoma, lymphoma, Burkitt lymphoma, Hodgkin lymphoma, myeloid leukemia, leukemia, sarcoma, blastoma, melanoma, seminoma, brain cancer, glioma, glioblastoma, cerebellar astrocytoma, cutaneous T-cell lymphoma, gastric cancer, liver cancer, ependymona, laryngeal cancer, neck cancer, stomach cancer, kidney cancer, pancreatic cancer, bladder cancer, esophageal cancer, testicular cancer, medulloblastoma, vaginal cancer, ovarian cancer, cervical cancer, basal cell carcinoma, pituitary adenoma, rhabdomyosarcoma, and Kaposi sarcoma.

7. A method for discovering antibodies, comprising:
identifying heavy chain immunoglobulin sequences in a target tissue from an animal;
identifying light chain immunoglobulin sequences in said target tissue from said animal; and
computationally pairing said identified single chain immunoglobulin sequences more frequent than 1% of clones with each said identified light chain immunoglobulin sequences more frequent than 1% of clones to obtain a candidate list of paired immunoglobulin sequences to which the target tissue may be antigenic.

8. The method of claim 7, wherein said tissue is a tissue sampled from an animal with a disease selected from the group consisting of: multiple sclerosis, rheumatoid arthritis, ulcerative colitis, Crohn's disease, systemic lupus erythematosus, graft-versus-host-disease, and host-versus-graft disease.

9. The method of claim 7, wherein said tissue is a tumor selected from the group consisting of: lung carcinoma, non-small cell lung cancer, small cell lung cancer, uterine cancer, thyroid cancer, breast carcinoma, prostate carcinoma, pancreas carcinoma, colon carcinoma, lymphoma, Burkitt lymphoma, Hodgkin lymphoma, myeloid leukemia, leukemia, sarcoma, blastoma, melanoma, seminoma, brain cancer, glioma, glioblastoma, cerebellar astrocytoma, cutaneous T-cell lymphoma, gastric cancer, liver cancer, ependymona, laryngeal cancer, neck cancer, stomach cancer, kidney cancer, pancreatic cancer, bladder cancer, esophageal cancer, testicular cancer, medulloblastoma, vaginal cancer, ovarian cancer, cervical cancer, basal cell carcinoma, pituitary adenoma, rhabdomyosarcoma, and Kaposi sarcoma.

10. A method for discovering antibodies, comprising:
identifying heavy and light chain immunoglobulin sequences in a target tissue from an animal with a disease or condition;
identifying heavy and light chain immunoglobulin sequences in said target tissue from an animal without said disease or condition;
obtaining a list of single heavy and light chain immunoglobulin sequences more frequent than 1% of clones in the animal with said disease or condition but not more frequent than 1% of clones in the animal without said disease or condition; and
computationally pairing heavy and light chain to obtain a candidate list of paired immunoglobulin sequences that may be associated with said disease or condition.

11. The method of claim 7, wherein the number of animals surveyed is 1.

12. The method of claim 7, wherein the number of animals surveyed is 10.

13. The method of claim 7, wherein the number of animals surveyed is 100.

14. The method of claim 7, wherein the number of animals surveyed is 1000.

15. The method of claim 7, wherein the number of animals surveyed is 10000.

16. The method of claim 7, wherein said tissue is a tissue sampled from an animal with a disease selected from the group consisting of: multiple sclerosis, rheumatoid arthritis, ulcerative colitis, Crohn's disease, systemic lupus erythematosus, graft-versus-host-disease, and host-versus-graft disease.

17. The method of claim 7, wherein said tissue is a tumor selected from the group consisting of: lung carcinoma, non-small cell lung cancer, small cell lung cancer, uterine cancer, thyroid cancer, breast carcinoma, prostate carcinoma, pancreas carcinoma, colon carcinoma, lymphoma, Burkitt lymphoma, Hodgkin lymphoma, myeloid leukemia, leukemia, sarcoma, blastoma, melanoma, seminoma, brain cancer, glioma, glioblastoma, cerebellar astrocytoma, cutaneous T-cell lymphoma, gastric cancer, liver cancer, ependymona, laryngeal cancer, neck cancer, stomach cancer, kidney cancer, pancreatic cancer, bladder cancer, esophageal cancer, testicular cancer, medulloblastoma, vaginal cancer, ovarian cancer, cervical cancer, basal cell carcinoma, pituitary adenoma, rhabdomyosarcoma, and Kaposi sarcoma.

18. The method of claim 1, wherein said tissue is a tissue sampled from an animal with an infectious disease.

19. The method of claim 1, wherein said tissue is a tissue sampled from an animal with an infectious disease.
